(19) United States
(12) Patent Application Publication
(10) Pub. No.: US 2024/0199582 A1
(43) Pub. Date: Jun. 20, 2024

(54) INHIBITORS OF POLY(ADP-RIBOSE) POLYMERASE

(71) Applicants: RHIZEN PHARMACEUTICALS AG, Basel (CH); Incozen Therapeutics Pvt. Ltd., Hyderabad (IN)

(72) Inventors: Swaroop Kumar Venkata Satya VAKKALANKA, Basel (CH); Debnath BHUNIYA, Hyderabad (IN); Srikant VISWANADHA, Hyderabad (IN)

(21) Appl. No.: 18/554,550

(22) PCT Filed: Apr. 7, 2022
(86) PCT No.: PCT/IB2022/053282

§ 371 Date: Oct. 9, 2023

(30) Foreign Application Priority Data
Apr. 8, 2021 (IN) 202141016598

(51) Int. Cl. C07D 401/14; A61K 31/502; A61K 45/06; A61P 35/00

(52) U.S. Cl. CPC C07D 401/14; A61K 31/502; A61K 45/06; A61P 35/00

(57) ABSTRACT

The present invention relates to Poly(ADP-ribose) polymerase (PARP) inhibitors, methods of preparing them, pharmaceutical compositions containing them and to their use in methods of treatment and/or prevention of PARP mediated diseases or disorders.

This application is the U.S. national phase of International Patent Application No. PCT/IB2022/053282, filed Apr. 7, 2022, which claims the benefit of Indian Provisional Patent Application No. 202141016598, filed on Apr. 8, 2021, the entire contents of which is hereby incorporated by reference.

### FIELD OF THE INVENTION

The present invention relates to poly(ADP-ribose) polymerase (PARP) inhibitors, pharmaceutical compositions containing them, methods of preparing them, and methods of treatment and/or prevention of PARP mediated diseases or disorders with them.

### BACKGROUND OF THE INVENTION

Poly (ADP-ribose) polymerases (PARPs) belong to a family of 18 members that catalyze the addition of ADP-ribose units to DNA or different acceptor proteins, which affect cellular processes as diverse as replication, transcription, differentiation, gene regulation, protein degradation and spindle maintenance. PARP-1 and PARP-2 are the two enzymes among the PARPs that have been extensively investigated, and research suggest that these two enzymes are activated by DNA damage and are involved in DNA repair. Loss of these two proteins leads to a tumor-specific dysfunction in the repair of double strand breaks by homologous recombination. PARP inhibitors (PARPi) were conceived as anticancer drugs based on the concept that if PARP enzymes did not repair DNA damage, this could lead cancer cells to develop too many mutations and trigger cell death. Four PARPi are currently approved for clinical use: olaparib, rucaparib, niraparib, and talazoparib. The advent of PARPi therapies is likely to have significant implications for the treatment of patients with cancer and hence there is an urgent need to develop new PARP inhibitors with more desirable efficacy and safety profiles.

International Publication No. WO 2021/220120 is incorporated herein by reference in its entirety.

### SUMMARY OF THE INVENTION

The present invention is directed to PARP inhibitors and salts (e.g., pharmaceutically acceptable salts) thereof. These compounds are suitable for use in the treatment of a PARP associated disease, disorder or condition, e.g., a proliferative disease such as cancer.

In one aspect, the present invention relates to 4-((5-(3-Hydroxy-3-methyl-2-oxoindolin-1-yl)pyridin-3-yl)methyl)phthalazin-1(2H)-one, or a pharmaceutically acceptable salt thereof.

In another aspect, the present invention relates to (R)-(+)-4-((5-(3-Hydroxy-3-methyl-2-oxoindolin-1-yl)pyridin-3-yl)methyl)phthalazin-1(2H)-one (Compound 1) or a pharmaceutically acceptable salt thereof.

In another aspect, the present invention relates to (S)-(−)-4-((5-(3-Hydroxy-3-methyl-2-oxoindolin-1-yl)pyridin-3-yl)methyl)phthalazin-1(2H)-one) (Compound 2) or a pharmaceutically acceptable salt thereof.

In one embodiment, the pharmaceutically acceptable salt of Compound 1 has an enantiomeric excess (e.e.) of at least about 70%, about 80%, about 90%, about 95%, about 98% or about 99%. In a further embodiment, Compound 1 is substantially free (e.g., contains less than about 30%, less than about 20%, less than about 10%, less than about 5% or less than about 1% by weight) or is free of Compound 2.

In one embodiment, the pharmaceutically acceptable salt of Compound 2 has an enantiomeric excess (e.e.) of at least about 70%, about 80%, about 90%, about 95%, about 98% or about 99%. In a further embodiment, Compound 2 is substantially free (e.g., contains less than about 30%, less than about 20%, less than about 10%, less than about 5% or less than about 1% by weight) or is free of Compound 1.

In still another embodiment, the present invention relates to a hydrochloride salt (e.g., a monohydrochloride salt) of (R)-(+)-4-((5-(3-Hydroxy-3-methyl-2-oxoindolin-1-yl)pyridin-3-yl)methyl)phthalazin-1(2H)-one (Compound 1A). Another embodiment is a crystalline hydrochloride salt (e.g., a monohydrochloride salt) of (R)-(+)-4-((5-(3-Hydroxy-3-methyl-2-oxoindolin-1-yl)pyridin-3-yl)methyl)phthalazin-1(2H)-one.

In yet another embodiment, the present invention relates to hydrochloride salt (e.g., a monohydrochloride salt) of (S)-(−)-4-((5-(3-Hydroxy-3-methyl-2-oxoindolin-1-yl)pyridin-3-yl)methyl)phthalazin-1(2H)-one (Compound 2A). Another embodiment is a crystalline hydrochloride salt (e.g., a monohydrochloride salt) of ((S)-(−)-4-((5-(3-Hydroxy-3-methyl-2-oxoindolin-1-yl)pyridin-3-yl)methyl)phthalazin-1(2H)-one.

In further embodiment, the present invention relates to a benzene sulfonate salt (e.g., a monobenzene sulfonate salt) of (R)-(+)-4-((5-(3-Hydroxy-3-methyl-2-oxoindolin-1-yl)pyridin-3-yl)methyl)phthalazin-1(2H)-one (Compound 1B). Another embodiment is a crystalline benzene sulfonate salt (e.g., a monobenzene sulfonate salt) of (R)-(+)-4-((5-(3-Hydroxy-3-methyl-2-oxoindolin-1-yl)pyridin-3-yl)methyl)phthalazin-1(2H)-one.

In a further embodiment, the present invention relates to a benzene sulfonate salt (e.g., a monobenzene sulfonate salt) of (S)-(−)-4-((5-(3-Hydroxy-3-methyl-2-oxoindolin-1-yl)pyridin-3-yl)methyl)phthalazin-1(2H)-one (Compound 2B). Another embodiment is a crystalline benzene sulfonate salt (e.g., a monobenzene sulfonate salt) of (S)-(−)-4-((5-(3-Hydroxy-3-methyl-2-oxoindolin-1-yl)pyridin-3-yl)methyl)phthalazin-1(2H)-one.

In still another embodiment, the present invention relates to a 4-methylbenzene sulfonate salt (PTSA) (e.g., a mono-4-methylbenzene sulfonate salt) of (R)-(+)-4-((5-(3-Hydroxy-3-methyl-2-oxoindolin-1-yl)pyridin-3-yl)methyl)phthalazin-1(2H)-one (Compound 1C). Another embodiment is a crystalline 4-methylbenzene sulfonate salt (PTSA) (e.g., a mono-4-methylbenzene sulfonate salt) of (R)-(+)-4-((5-(3-Hydroxy-3-methyl-2-oxoindolin-1-yl)pyridin-3-yl)methyl)phthalazin-1(2H)-one.

In yet another embodiment, the present invention relates to a 4-methyl benzene sulfonate salt (PTSA) (e.g., a mono-4-methylbenzene sulfonate salt) of (S)-(−)-4-((5-(3-Hydroxy-3-methyl-2-oxoindolin-1-yl)pyridin-3-yl)methyl)phthalazin-1(2H)-one (Compound 2C). Another embodiment is a crystalline 4-methyl benzene sulfonate salt (PTSA) (e.g., a mono-4-methylbenzene sulfonate salt) of (S)-(−)-4-((5-(3-Hydroxy-3-methyl-2-oxoindolin-1-yl)pyridin-3-yl)methyl)phthalazin-1(2H)-one.

In still a further embodiment, the present invention relates to a methane sulfonate salt (e.g., a mono-methane sulfonate salt) of (R)-(+)-4-((5-(3-Hydroxy-3-methyl-2-oxoindolin-1-yl)pyridin-3-yl)methyl)phthalazin-1(2H)-one (Compound 1D). Another embodiment is a crystalline methane sulfonate salt (e.g., a mono-methane sulfonate salt) of (R)-(+)-4-((5-(3-Hydroxy-3-methyl-2-oxoindolin-1-yl)pyridin-3-yl)methyl)phthalazin-1(2H)-one.

In a further embodiment, the present invention relates to a methane sulfonate salt (e.g., a mono-methane sulfonate salt) of (S)-(−)-4-((5-(3-Hydroxy-3-methyl-2-oxoindolin-1-yl)pyridin-3-yl)methyl)phthalazin-1(2H)-one (Compound 2D). Another embodiment is a crystalline methane sulfonate salt (e.g., a mono-methane sulfonate salt) of (S)-(−)-4-((5-(3-Hydroxy-3-methyl-2-oxoindolin-1-yl)pyridin-3-yl)methyl)phthalazin-1(2H)-one.

The chemical structures of Compounds 1 and 2 are shown below.

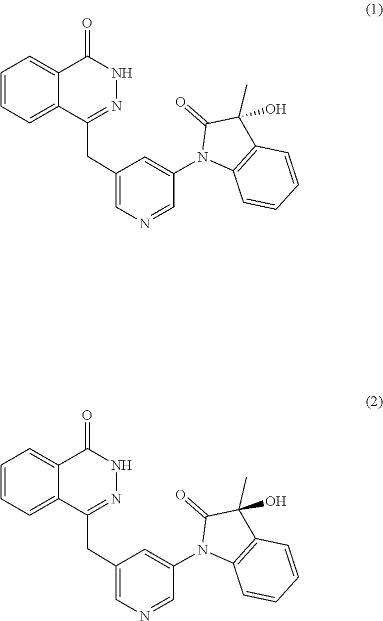 [SMILES: C[C@]1(O)C(=O)N(c2cncc(Cc3n[nH]c(=O)c4ccccc34)c2)c2ccccc21 ; C[C@@]1(O)C(=O)N(c2cncc(Cc3n[nH]c(=O)c4ccccc34)c2)c2ccccc21]

The present invention further provides a pharmaceutical composition comprising one or more compounds of any embodiment of the present invention (e.g., Compound 1 and/or Compound 2, and pharmaceutically acceptable salts thereof, or mixtures thereof) and a pharmaceutically acceptable carrier. The pharmaceutical composition may further comprise one or more additional active ingredients. In one embodiment, the pharmaceutical composition includes a therapeutically effective amount of one or more compounds of any embodiment of the present invention.

The present invention further provides a pharmaceutical composition comprising a pharmaceutically acceptable salt of Compound 1 and a pharmaceutically acceptable carrier.

The present invention further provides a pharmaceutical composition comprising a pharmaceutically acceptable salt of Compound 2 and a pharmaceutically acceptable carrier.

In one embodiment, the present invention provides a pharmaceutical composition comprising Compound 1, or a pharmaceutically acceptable salt thereof, wherein Compound 1 (or its pharmaceutically acceptable salt) is present in an enantiomeric excess of Compound 2 (or its pharmaceutically acceptable salt), for example, Compound 1 has an enantiomeric excess (e.e.) of at least about 70%, about 80%, about 90%, about 95%, about 98% or about 99%. In one embodiment, Compound 1 (or its pharmaceutically acceptable salt) is substantially free (e.g., contains less than about 30%, less than about 20%, less than about 10%, less than about 5% or less than about 1% by weight or is free of Compound 2 (or its pharmaceutically acceptable salt) in the pharmaceutical composition.

In one embodiment, the present invention provides a pharmaceutical composition comprising Compound 2, or a pharmaceutically acceptable salt thereof, wherein Compound 2 (or its pharmaceutically acceptable salt) is present in an enantiomeric excess of Compound 1 (or its pharmaceutically acceptable salt), for example Compound 2 (or its pharmaceutically acceptable salt) has an enantiomeric excess (e.e.) of at least about 70%, about 80%, about 90%, about 95%, about 98% or about 99%. In one embodiment, Compound 2 (or its pharmaceutically acceptable salt) is substantially free (e.g., contains less than about 30%, less than about 20%, less than about 10%, less than about 5% or less than about 1% by weight or is free of Compound 1 (or its pharmaceutically acceptable salt) in the pharmaceutical composition.

In one embodiment, the methane sulfonate salt (e.g., the mono-methane sulfonate salt) of Compound 1 has an enantiomeric excess (e.e.) of at least about 70%, about 80%, about 90%, about 95%, about 98% or about 99%.

In one embodiment, the PTSA salt (e.g., the mono-PTSA salt) of Compound 1 has an enantiomeric excess (e.e.) of at least about 70%, about 80%, about 90%, about 95%, about 98% or about 99%.

In one embodiment, the benzenesulfonate salt (e.g., the mono-benzenesulfonate salt) of Compound 1 has an enantiomeric excess (e.e.) of at least about 70%, about 80%, about 90%, about 95%, about 98% or about 99%.

In one embodiment, the hydrochloride salt (e.g., the mono-hydrochloride salt) of Compound 1 has an enantiomeric excess (e.e.) of at least about 70%, about 80%, about 90%, about 95%, about 98% or about 99%.

In one embodiment, the methane sulfonate salt (e.g., the mono-methane sulfonate salt) of Compound 2 has an enantiomeric excess (e.e.) of at least about 70%, about 80%, about 90%, about 95%, about 98% or about 99%.

In one embodiment, the PTSA salt (e.g., the mono-PTSA salt) of Compound 2 has an enantiomeric excess (e.e.) of at least about 70%, about 80%, about 90%, about 95%, about 98% or about 99%.

In one embodiment, the benzenesulfonate salt (e.g., the mono-benzenesulfonate salt) of Compound 2 has an enantiomeric excess (e.e.) of at least about 70%, about 80%, about 90%, about 95%, about 98% or about 99%.

In one embodiment, the hydrochloride salt (e.g., the mono-hydrochloride salt) of Compound 2 has an enantiomeric excess (e.e.) of at least about 70%, about 80%, about 90%, about 95%, about 98% or about 99%.

In yet another embodiment, the hydrochloride salt (e.g., the mono-hydrochloride salt) of Compound 1 exhibits a differential scanning calorimeter (DSC) pattern having a characteristic endothermic peak at range of about 223° C. to about 232° C. (e.g., about 228° C. or about 228.3° C.).

In yet another embodiment, the hydrochloride salt (e.g., the mono-hydrochloride salt) of Compound 1 exhibits a DSC pattern having a characteristic endothermic peak at about 228° C. (e.g., about 228.3° C. or about 228.33° C.). In yet another embodiment, the hydrochloride salt (e.g., the mono-hydrochloride salt) of Compound 1 exhibits a DSC pattern having a characteristic endothermic peak at about 228° C. (e.g., about 228.3° C. or about 228.33° C.) with a Δ enthalpy of about 67.36 J/g.

In yet another embodiment, the hydrochloride salt (e.g., the mono-hydrochloride salt) of Compound 1 exhibits a DSC thermogram substantially as depicted in FIG. 1.

In yet another embodiment, the benzene sulfonate salt (e.g., the mono-benzene sulfonate salt) of Compound 1 exhibits a DSC pattern having a characteristic endothermic peak at range of about 226° C. to 236° C. (e.g., about 231° C. or about 231.5° C.).

In yet another embodiment, the benzene sulfonate salt (e.g., the mono-benzene sulfonate salt) of Compound 1 exhibits a DSC pattern having a characteristic endothermic peak at about 231° C. In yet another embodiment, the benzene sulfonate salt (e.g., the mono-benzene sulfonate salt) of Compound 1 exhibits a DSC pattern having a characteristic endothermic peak at about 231° C. (e.g., about 231.5° C. or about 231.48° C.) with a Δ enthalpy of about 83.04 J/g.

In yet another embodiment, the benzene sulfonate salt (e.g., the mono-benzene sulfonate salt) of Compound 1 exhibits a DSC thermogram substantially as depicted in FIG. 2.

In yet another embodiment, the methylbenzene sulfonate salt (e.g., the mono-methylbenzene sulfonate salt) of Compound 1 (for example, prepared by using Method 2 of Example 1C) exhibits a DSC pattern having a characteristic endothermic peak at range of about 165° C. to about 175° C. (e.g., about 170° C. or about 170.2° C.).

In yet another embodiment, the methylbenzene sulfonate salt (e.g., the mono-methylbenzene sulfonate salt) of Compound 1 exhibits a DSC pattern having a characteristic endothermic peak at about 170° C. (e.g., about 170.2° C. or about 170.24° C.). In yet another embodiment, the methylbenzene sulfonate salt (e.g., the mono-methylbenzene sulfonate salt) of Compound 1 exhibits a DSC pattern having a characteristic endothermic peak at about 170° C. (e.g., about 170.2° C. or about 170.24° C.) with a Δ enthalpy of 55 J/g (e.g., about 55.16 J/g).

In yet another embodiment, the methylbenzene sulfonate salt (e.g., the mono-methylbenzene sulfonate salt) of Compound 1 exhibits a DSC thermogram substantially as depicted in FIG. 3.

In yet another embodiment, the methane sulfonate salt (e.g., the mono-methane sulfonate salt) of Compound 1 (e.g., prepared by Method 1, 2, or 4 of Example 1D) exhibits a DSC pattern having a characteristic endothermic peak in the range of about 190° C. to about 220° C.

In yet another embodiment, the methane sulfonate salt (e.g., the mono-methane sulfonate salt) of Compound 1 (e.g., prepared by Method 3) exhibits a DSC pattern having a characteristic endothermic peak in the range of about 165° C. to about 175° C.

In yet another embodiment, the methane sulfonate salt (e.g., the mono-methane sulfonate salt) of Compound 1 exhibits a DSC thermogram substantially as depicted in FIG. 4A, FIG. 4B, FIG. 4C or FIG. 4D.

In yet another embodiment, the methane sulfonate salt (e.g., the mono-methane sulfonate salt) of Compound 1 exhibits a DSC thermogram substantially as depicted in FIG. 4A.

In yet another embodiment, the methane sulfonate salt (e.g., the mono-methane sulfonate salt) of Compound 1 exhibits a DSC thermogram substantially as depicted in FIG. 4B.

In yet another embodiment, the methane sulfonate salt (e.g., the mono-methane sulfonate salt) of Compound 1 exhibits a DSC thermogram substantially as depicted in FIG. 4C.

In yet another embodiment, the methane sulfonate salt (e.g., the mono-methane sulfonate salt) of Compound 1 exhibits a DSC thermogram substantially as depicted in FIG. 4D.

In yet another embodiment, the methane sulfonate salt (e.g., the mono-methane sulfonate salt) of Compound 1 prepared according to Method 1 of Example 1D, exhibits a DSC pattern having a characteristic endothermic peak of about 205° C. (e.g., about 204.9° C. or about 204.94° C.).

In yet another embodiment, the methane sulfonate salt (e.g., the mono-methane sulfonate salt) of Compound 1, prepared according to Method 1 of Example 1D, exhibits a DSC thermogram substantially as depicted in FIG. 4A.

In yet another embodiment, the methane sulfonate salt (e.g., the mono-methane sulfonate salt) of Compound 1, prepared according to Method 2 of Example 1D, exhibits a DSC pattern having a characteristic endothermic peak of about 208° C. (e.g., about 208.2° C. or about 208.24° C.).

In yet another embodiment, the methane sulfonate salt (e.g., the mono-methane sulfonate salt) of Compound 1, prepared according to Method 2 of Example 1D, exhibits a DSC thermogram substantially as depicted in FIG. 4B.

In yet another embodiment, the methane sulfonate salt (e.g., the mono-methane sulfonate salt) of Compound 1, prepared according to Method 3 of Example 1D, exhibits a DSC pattern having a characteristic endothermic peak of about 171° C. (e.g., about 171.8° C.).

In yet another embodiment, the methane sulfonate salt (e.g., the mono-methane sulfonate salt) of Compound 1, prepared according to Method 3 of Example 1D, exhibits a DSC thermogram substantially as depicted in FIG. 4C.

In yet another embodiment, the methane sulfonate salt (e.g., the mono-methane sulfonate salt) of Compound 1, prepared according to Method 4 of Example 1D, exhibits a DSC pattern having a characteristic endothermic peak of about 210° C. (e.g., about 210.28° C.).

In yet another embodiment, the methane sulfonate salt (e.g., the mono-methane sulfonate salt) of Compound 1, prepared according to Method 4 of Example 1D, exhibits a DSC thermogram substantially as depicted in FIG. 4D.

In yet another embodiment, the hydrochloride salt (e.g., the mono-hydrochloride salt) of Compound 1 exhibits an X-ray powder diffraction (XRPD) pattern exhibiting one or more (such as 1, 2, 3, 4, 5, 6, 7 or 8) characteristic peaks at 5.32, 10.65, 14.91, 15.22, 16.68, 19.90, 21.75, 21.99, 23.84, 25.08, 27.14±0.05, 0.1, or 0.2° 2θ.

In yet another embodiment, the hydrochloride salt (e.g., the mono-hydrochloride salt) of Compound 1 exhibits an XRPD pattern substantially as depicted in FIG. 5.

In yet another embodiment, the hydrochloride salt (e.g., the mono-hydrochloride salt) of Compound 1 exhibits an X-ray powder diffraction (XRPD) pattern exhibiting one or more (such as 1, 2, 3, 4, 5, 6, 7 or 8) characteristic peaks at 5.32, 10.65, 14.91, 15.22, 16.68, 19.90, 21.75, 21.99, 23.84, 25.08, 27.14±0.05, 0.1, or 0.2° 2θ; and a differential scanning calorimeter (DSC) pattern having a characteristic endothermic peak at range of about 226° C. to 236° C. (e.g., about 231° C., about 231.5° C., or about 231.48° C.).

In yet another embodiment, the benzene sulfonate salt (e.g., the mono-benzene sulfonate salt) of Compound 1 exhibits an XRPD pattern exhibiting one or more (such as 1, 2, 3, 4, 5, 6, 7 or 8) characteristic peaks at 4.91, 5.42, 13.76, 14.61, 18.47, 21.14, 22.19, 23.07, 23.84, 25.28±0.05, 0.1, or 0.2° 2θ.

In yet another embodiment, the benzene sulfonate salt (e.g., the mono-benzene sulfonate salt) of Compound 1 exhibits an XRPD pattern substantially as depicted in FIG. 6.

In yet another embodiment, the benzene sulfonate salt (e.g., the mono-benzene sulfonate salt) of Compound 1 exhibits an XRPD pattern exhibiting one or more (such as 1, 2, 3, 4, 5, 6, 7 or 8) characteristic peaks at 4.91, 5.42, 13.76, 14.61, 18.47, 21.14, 22.19, 23.07, 23.84, 25.28±0.05, 0.1, or 0.2° 2θ; and a differential scanning calorimeter (DSC) pattern having a characteristic endothermic peak at range of about 226° C. to 236° C. (e.g., about 231° C., about 231.5° C., or about 231.48° C.).

In yet another embodiment, the methylbenzene sulfonate salt (e.g., the mono-methylbenzene sulfonate salt) of Compound 1 (for example, prepared by Method 1 of Example 1C) exhibits an XRPD pattern exhibiting one or more (such as 1, 2, 3, 4, 5, 6, 7 or 8) characteristic peaks at 6.81, 13.22, 13.96, 20.52, 21.87, 22.67, 24.48±0.05, 0.1, or 0.2° 2θ.

In yet another embodiment, the methylbenzene sulfonate salt (e.g., the mono-methylbenzene sulfonate salt) of Compound 1 (for example, prepared by using Method 1 of Example 1C) exhibits an XRPD pattern substantially as depicted in FIG. 7A.

In yet another embodiment, the methylbenzene sulfonate salt (e.g., the mono-methylbenzene sulfonate salt) of Compound 1 (for example, prepared by using Method 2 of Example 1C) exhibits an XRPD pattern exhibiting one or more (such as 1, 2, 3, 4, 5, 6, 7 or 8) peaks selected from 6.98, 13.82, 15.98, 18.50, 19.50±0.05, 0.1, or 0.2° 2θ.

In yet another embodiment, the methylbenzene sulfonate salt (e.g., the mono-methylbenzene sulfonate salt) of Compound 1 (for example, prepared by using Method 2 of Example 1C) exhibits an XRPD pattern substantially as depicted in FIG. 7B.

In yet another embodiment, the methylbenzene sulfonate salt (e.g., the mono-methylbenzene sulfonate salt) of Compound 1 (for example, prepared by using Method 2) exhibits an XRPD pattern exhibiting one or more (such as 1, 2, 3, 4, 5, 6, 7 or 8) peaks selected from 6.98, 13.82, 15.98, 18.50, 19.50±0.05, 0.1, or 0.2° 2θ, and a differential scanning calorimeter (DSC) pattern having a characteristic endothermic peak at range of about 165° C. to 175° C. (e.g., about 170° C., about 170.2° C., or about 170.24).

In yet another embodiment, the methane sulfonate salt (e.g., the mono-methane sulfonate salt) of Compound 1 (for example, prepared by using Method 1 of Example 1D) exhibits an XRPD pattern exhibiting one or more (such as 1, 2, 3, 4, 5, 6, 7 or 8 peaks) characteristic peaks at 5.84, 11.17, 13.78, 14.60, 19.17, 20.03, 21.32, 22.24, 22.77, 26.40±0.05, 0.1, or 0.2° 2θ. In certain embodiments, the crystalline methane sulfonate salt (e.g., the mono-methane sulfonate salt) of Compound 1 is substantially free (e.g., contains less than about 30%, less than about 20%, less than about 10%, less than about 5% or less than about 1% by weight) or is free of other crystalline forms of the methane sulfonate salt (e.g., the mono-methane sulfonate salt) of Compound 1.

In yet another embodiment, the methane sulfonate salt (e.g., the mono-methane sulfonate salt) of Compound 1 exhibits an XRPD pattern substantially as depicted in FIG. 8A.

In yet another embodiment, the methane sulfonate salt (e.g., the mono-methane sulfonate salt) of Compound 1 (for example, prepared by using Method 1 of Example 1D) exhibits an XRPD pattern exhibiting one or more (such as 1, 2, 3, 4, 5, 6, 7 or 8 peaks) characteristic peaks at 5.84, 11.17, 13.78, 14.60, 19.17, 20.03, 21.32, 22.24, 22.77, 26.40±0.05, 0.1, or 0.2° 2θ; and a differential scanning calorimeter (DSC) pattern having a characteristic endothermic peak of about 205° C. (e.g., about 204.9° C. or about 204.94° C.).

In yet another embodiment, the methane sulfonate salt (e.g., the mono-methane sulfonate salt) of Compound 1 (for example, prepared by using Method 2 of Example 1D) exhibits an XRPD pattern exhibiting one or more (such as 1, 2, 3, 4, 5, 6, 7 or 8 peaks) characteristic peaks at 5.74, 11.20. 13.69, 14.67, 19.20, 20.05, 21.29, 22.57, 26.38±0.05, 0.1, or 0.2° 2θ. In certain embodiments this crystalline methane sulfonate salt (e.g., the mono-methane sulfonate salt) of Compound 1 is substantially free (e.g., contains less than about 30%, less than about 20%, less than about 10%, less than about 5% or less than about 1% by weight) or is free of other crystalline forms of the methane sulfonate salt (e.g., the mono-methane sulfonate salt) Compound 1.

In yet another embodiment, the methane sulfonate salt (e.g., the mono-methane sulfonate salt) of Compound 1 exhibits an XRPD pattern substantially as depicted in FIG. 8B.

In yet another embodiment, the methane sulfonate salt (e.g., the mono-methane sulfonate salt) of Compound 1 (for example, prepared by using Method 2 of Example 1D) exhibits an XRPD pattern exhibiting one or more (such as 1, 2, 3, 4, 5, 6, 7 or 8 peaks) characteristic peaks at 5.74, 11.20. 13.69, 14.67, 19.20, 20.05, 21.29, 22.57, 26.38±0.05, 0.1, or 0.2° 2θ; and a differential scanning calorimeter (DSC) pattern having a characteristic endothermic peak of about 208° C. (e.g., about 208.2° C. or about 208.24° C.).

In yet another embodiment, the methane sulfonate salt (e.g., the mono-methane sulfonate salt) of Compound 1 (for example, prepared by using Method 3 of Example 1D) exhibits an XRPD pattern exhibiting one or more (such as 1, 2, 3, 4, 5, 6, 7 or 8 peaks) characteristic peaks at 5.73, 11.02, 11.19, 13.68, 14.55, 15.11, 19.11, 20.04, 21.28, 22.19, 22.65, 26.15±0.05, 0.1, or 0.2° 2θ. In certain embodiments, the crystalline methane sulfonate salt (e.g., the mono-methane sulfonate salt) of Compound 1 is substantially free (e.g., contains less than about 30%, less than about 20%, less than about 10%, less than about 5% or less than about 1% by weight) or is free of other crystalline forms of the methane sulfonate salt (e.g., the mono-methane sulfonate salt) of Compound 1.

In yet another embodiment, the methane sulfonate salt (e.g., the mono-methane sulfonate salt) of Compound 1 exhibits an XRPD pattern substantially as depicted in FIG. 8C.

In yet another embodiment, the methane sulfonate salt (e.g., the mono-methane sulfonate salt) of Compound 1 (for example, prepared by using Method 3 of Example 1D) exhibits an XRPD pattern exhibiting one or more (such as 1, 2, 3, 4, 5, 6, 7 or 8 peaks) characteristic peaks at 5.73, 11.02, 11.19, 13.68, 14.55, 15.11, 19.11, 20.04, 21.28, 22.19, 22.65, 26.15±0.05, 0.1, or 0.2° 2θ; and a differential scanning calorimeter (DSC) pattern having a characteristic endothermic peak of about 171° C. (e.g., about 171.8° C.).

In yet another embodiment, the methane sulfonate salt (e.g., the mono-methane sulfonate salt) of Compound 1 (for example, prepared by using Method 4 of Example 1D) exhibits an XRPD pattern exhibiting one or more (such as 1, 2, 3, 4, 5, 6, 7 or 8) characteristic peaks at 5.67, 10.81, 14.34, 19.03, 20.40, 21.96, 23.44, 24.52, 25.94±0.05, 0.1, or 0.2° 2θ.

In yet another embodiment, the methane sulfonate salt of Compound 1 (e.g., the mono-methane sulfonate salt) of Compound 1 (for example, prepared by using Method 4 of Example 1D) exhibits an XRPD pattern substantially as depicted in FIG. 8D.

In yet another embodiment, the methane sulfonate salt (e.g., the mono-methane sulfonate salt) of Compound 1 (for example, prepared by using Method 4 of Example 1D) exhibits an XRPD pattern exhibiting one or more (such as 1, 2, 3, 4, 5, 6, 7 or 8) characteristic peaks at 5.67, 10.81, 14.34, 19.03, 20.40, 21.96, 23.44, 24.52, 25.94±0.05, 0.1, or 0.2° 2θ; and a differential scanning calorimeter (DSC) pattern having a characteristic endothermic peak of about 210° C. (e.g., about 210.28° C.).

Another aspect of the present invention is directed to a method for preparing the methane sulfonate, 4-methylbenzenesulfonate (PTSA), benzenesulfonate or hydrochloride salt of Compound 1 or Compound 2. In one embodiment, the method comprises converting Compound 1 or Compound 2, or a salt thereof (other than the desired salt) to a methane sulfonate, 4-methylbenzenesulfonate, benzenesulfonate, or hydrochloride salt of Compound 1 or Compound 2.

Another aspect of the present invention is directed to a method for preparing a methane sulfonate, 4-methylbenzenesulfonate (PTSA), benzenesulfonate or hydrochloride salt of Compound 1 or Compound 2. In one embodiment, the method comprises converting Compound 1 or Compound 2, or a salt thereof (other than the desired salt) to a methane sulfonate, 4-methylbenzenesulfonate, benzenesulfonate, or hydrochloride salt of Compound 1 or Compound 2 in the presence of a suitable solvent selected from, e.g., acetone, methyl ethyl ketone, methyl isobutyl ketone, methyl amyl ketone, cyclohexanone, isophorone, di isobutyl ketone, and di acetone alcohol.

Also provided is a methane sulfonate, 4-methylbenzenesulfonate (PTSA), benzenesulfonate or hydrochloride salt of Compound 1 or Compound 2 obtained by any of the methods for preparing the salt disclosed herein.

In yet another embodiment, exemplary methods for preparing salts of Compound 1 and Compound 2 are as described in Table 1.

TABLE 1

| | | Melting |
|---|---|---|
| | | Point* of Salt |
| Salt | Method of Preparation | (° C.) |
| Hydrochloride | Compound 1 was suspended in methanol. To this mixture | 226-228 |
| | concentrated hydrochloric acid was added, and the mixture | |
| | was stirred to obtain a solid. At room temperature, methyl | |
| | tert-butyl ether was then added. The reaction mixture was | |
| | stirred and the resulting solid was filtered and dried. | |
| Benzene | Compound 1 was suspended in acetone. Benzenesulfonic | 130-134 |
| sulfonate | acid was dissolved in acetone and then added to the | |
| | suspension. This mixture was then stirred at temperature of | |
| | about 50° C. ± 10° C., cooled to room temperature and diluted | |
| | with diisopropyl ether to obtain a solid product. This mixture | |
| | was further stirred at room temperature, and the resulting | |
| | solid was filtered, washed with diisopropyl ether and dried | |
| | in vacuo. | |
| 4- | Compound 1 was suspended in acetone/methanol. p- | 148-176 |
| methylbenzene | Toluene sulfonic acid monohydrate, dissolved in | |
| sulfonate | acetone/methanol, was then added to the suspension. The | |
| | resulting heterogeneous mixture was stirred at room | |
| | temperature and at about 70° C. ± 10° C. The reaction mixture | |
| | was then cooled to room temperature with stirring, then | |
| | diluted with diisopropyl ether or methyl tert-butyl ether to | |
| | obtain a solid, which was washed with diisopropyl ether or | |
| | methyl tert-butyl ether, respectively, then dried. | |
| Methane | Compound 1 or Compound 2 was suspended in acetone. | 190-198 |
| sulfonate | Methanesulfonic acid was then added to the suspension. | |
| | This resulting mixture was stirred at room temperature and | |
| | at about 50° C. ± 10° C. After cooling, the mixture was then | |
| | diluted with diisopropyl ether. The resulting solid was then | |
| | filtered, washed with diisopropyl ether/cyclohexane and | |
| | dried. | |

*The melting points disclosed in the table above were physically determined using a capillary method. The salt was taken into the capillary tube and heated until completely melted.

Another embodiment of the present invention relates to a methane sulfonate, 4-methylbenzenesulfonate (PTSA), benzenesulfonate, or hydrochloride salt of Compound 1 or Compound 2 according to any embodiment described herein, for use as a medicament.

Another embodiment of the present invention relates to a methane sulfonate, 4-methylbenzenesulfonate (PTSA), benzenesulfonate, or hydrochloride salt of Compound 1 or Compound 2 according to any embodiment described herein, for use the treatment of a PARP associated disease, disorder or condition, e.g., a proliferative disease such as cancer.

Another embodiment of the present invention relates to a methane sulfonate, 4-methylbenzenesulfonate (PTSA), benzenesulfonate, or hydrochloride salt of Compound 1 or Compound 2 according to any embodiment described herein, for use in a pharmaceutical composition for the treatment of a PARP associated disease, disorder or condition, e.g., a proliferative disease such as cancer.

Another embodiment of the present invention relates to the use of a methane sulfonate, 4-methylbenzenesulfonate (PTSA), benzenesulfonate, or hydrochloride salt of Compound 1 or Compound 2 according to any embodiment described herein, for the manufacture of a medicament for the treatment of a PARP associated disease, disorder or condition, e.g., a proliferative disease such as cancer.

The present invention further provides a pharmaceutical composition comprising a methane sulfonate, 4-methylbenzenesulfonate (PTSA), benzenesulfonate, or hydrochloride salt of Compound 1 according to any embodiment described herein, and a pharmaceutically acceptable carrier. The pharmaceutical composition may further comprise one or more of additional active ingredients.

The present invention further provides a pharmaceutical composition comprising a methane sulfonate, 4-methylbenzenesulfonate (PTSA), benzenesulfonate or hydrochloride salt of Compound 2 according to any embodiment described herein, and a pharmaceutically acceptable carrier. The pharmaceutical composition may further comprise one or more of additional active ingredients.

The present invention further provides a method of inhibiting PARP in a subject (e.g., a subject in need thereof) comprising administering to the subject an effective amount of a pharmaceutically acceptable salt of Compound 1 or a pharmaceutically acceptable salt of Compound 2, according to any embodiment described herein.

Yet another embodiment is a method of treating, preventing, and/or inhibiting a PARP mediated disease, disorder or condition (such as cancer or other proliferative disease or disorder) in a subject (e.g., a subject in need thereof) comprising administering to the subject an effective amount of a compound of the present invention according to any embodiment described herein.

In one embodiment, the amount of the compound (e.g., a pharmaceutically acceptable salt of Compound 1 or a pharmaceutically acceptable salt of Compound 2, according to any embodiment described herein) administered is sufficient to treat a PARP associated disease, disorder or condition by inhibition of PARP.

Yet another embodiment of the present invention is a method for treating a proliferative disease comprising administering to a subject (e.g., a subject in need thereof) an effective amount of at least one compound of the present invention according to any embodiment described herein (e.g., a pharmaceutically acceptable salt of Compound 1 or a pharmaceutically acceptable salt of Compound 2, according to any embodiment described herein). In one embodiment, the amount of the compound (e.g., a pharmaceutically acceptable salt of Compound 1 or a pharmaceutically acceptable salt of Compound 2, according to any embodiment described herein) administered is sufficient to treat the proliferative disease by inhibition of PARP

Yet another embodiment of the present invention is a method for treating a proliferative disease by administering to a subject (e.g., a subject in need thereof) an effective amount of at least one compound of the present invention (e.g., a pharmaceutically acceptable salt of Compound 1 or a pharmaceutically acceptable salt of Compound 2, according to any embodiment described herein) in combination (simultaneously or sequentially) with at least one other anti-cancer agent. In one embodiment, the amount of the compound (e.g., a pharmaceutically acceptable salt of Compound 1 or a pharmaceutically acceptable salt of Compound 2, according to any embodiment described herein) administered is sufficient to treat (or facilitate the treatment of) the proliferative disease by inhibition of PARP.

Yet another embodiment is a method of treating a PARP associated disease, disorder or condition in a subject (e.g., a subject in need thereof) comprising administering to the subject a pharmaceutical composition comprising a compound of any of the embodiments described herein (e.g., a pharmaceutically acceptable salt of Compound 1 or a pharmaceutically acceptable salt of Compound 2, according to any embodiment described herein) optionally admixed with at least one pharmaceutically acceptable excipient. In certain embodiments, the composition comprises a therapeutically effective amount of a compound of any of any of the embodiments described herein (e.g., a pharmaceutically acceptable salt of Compound 1 or a pharmaceutically acceptable salt of Compound 2, according to any embodiment described herein) for the treatment of PARP associated disease, disorder or condition.

Additional embodiments provide a method of treating cancer in a subject (e.g., a subject in need thereof) comprising administering to the subject a pharmaceutical composition comprising a compound of any of the embodiments described herein (e.g., a pharmaceutically acceptable salt of Compound 1 or a pharmaceutically acceptable salt of Compound 2, according to any embodiment described herein), optionally admixed with at least one pharmaceutically acceptable excipient. In certain embodiments, the composition comprises a therapeutically effective amount of compound of any of the embodiments described herein (e.g., a pharmaceutically acceptable salt of Compound 1 or a pharmaceutically acceptable salt of Compound 2, according to any embodiment described herein) for the treatment of cancer.

The compounds described herein are useful in the treatment of a variety of cancers, including, but not limited to:
carcinoma, including that of the bladder, breast, colon, kidney, liver, lung (including small cell lung cancer), esophagus, gall bladder, uterus, ovary, testes, larynx, oral cavity, gastrointestinal tract (e.g., esophagus, stomach, pancreas), brain, cervix, thyroid, prostate, blood, and skin (including squamous cell carcinoma);
hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma;
hematopoietic tumors of myeloid lineage, including acute and chronic myelogenous leukemias, myelodysplastic syndrome and promyelocytic leukemia;
tumors of mesenchymal origin, including fibrosarcoma and rhabdomyosarcoma;
tumors of the central and peripheral nervous system, including astrocytoma, neuroblastoma, glioma and schwannomas; and
other tumors, including melanoma, seminoma, teratocarcinoma, osteosarcoma, xenoderoma pigmentosum, keratoctanthoma, thyroid follicular cancer and Kaposi's sarcoma.

The compounds described herein, as modulators of apoptosis, are useful in the treatment of cancer (including but not limited to those types mentioned herein above), viral infections (including but not limited to herpevirus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), myelodysplastic syndromes, aplastic anaemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anaemia), degenerative diseases of the musculoskeletal system (including but not limited to osteoporosis and arthritis) aspirin-sensitive rhinosinusitis, cystic fibrosis, multiple sclerosis, kidney diseases and cancer pain.

The compounds described herein modulate the level of cellular RNA and DNA synthesis. The compounds described herein are therefore useful in the treatment of viral infections (including but not limited to HIV, human papilloma virus, herpesvirus, poxvirus, Epstein-Barr virus, Sindbis virus and adenovirus).

The compounds described herein are useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse. The compounds described herein are also useful in inhibiting tumor angiogenesis and metastasis. One embodiment of the invention is a method of inhibiting tumor angiogenesis or metastasis in a patient in need thereof by administering an effective amount of one or more compounds of the present invention.

Another embodiment of the present invention is a method of treating an immune system-related disease (e.g., an autoimmune disease), a disease or disorder involving inflammation (e.g., asthma, chronic obstructive pulmonary disease, rheumatoid arthritis, inflammatory bowel disease, glomerulonephritis, neuroinflammatory diseases, multiple sclerosis, uveitis and disorders of the immune system), cancer or other proliferative disease, a hepatic disease or disorder, or a renal disease or disorder. The method includes administering an effective amount of one or more compounds described herein.

Examples of immune disorders include, but are not limited to, psoriasis, rheumatoid arthritis, vasculitis, inflammatory bowel disease, dermatitis, osteoarthritis, asthma, inflammatory muscle disease, allergic disease (e.g., allergic rhinitis), vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, multiple sclerosis, cystic fibrosis, chronic relapsing hepatitis, primary biliary cirrhosis, allergic conjunctivitis and atopic dermatitis.

In one embodiment, the compounds described herein are used as immunosuppressants to prevent transplant graft rejections, allogeneic or xenogeneic transplantation rejection (organ, bone marrow, stem cells, other cells and tissues), and graft-versus-host disease. In other embodiments, transplant graft rejections result from tissue or organ transplants. In further embodiments, graft-versus-host disease results from bone marrow or stem cell transplantation. One embodiment is a method of preventing or decreasing the risk of transplant graft rejection, allogeneic or xenogeneic transplantation rejection (organ, bone marrow, stem cells, other cells and tissues), or graft-versus-host disease by administering an effective amount of one or more compounds of the present invention.

The compounds described herein are also useful in combination (administered together or sequentially) with known anti-cancer treatments, such as, but not limited to, radiation therapy or with cytostatic, cytotoxic or anticancer agents, such as for example, but not limited to, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate, other tyrosine kinase inhibitors such as Iressa and OSI-774; angiogenesis inhibitors; EGF inhibitors; VEGF inhibitors; CDK inhibitors; HDAC inhibitors, SRC inhibitors; c-Kit inhibitors; Her1/2 inhibitors and monoclonal antibodies directed against growth factor receptors such as erbitux (EGF) and herceptin (Her2) and other protein kinase modulators as well.

The compounds described herein are also useful in combination (administered together or sequentially) with one or more steroidal, anti-inflammatory drugs, non-steroidal anti-inflammatory drugs (NSAIDs) or immune selective anti-inflammatory derivatives (ImSAIDs).

Yet another embodiment is a method of treating cancer in a patient in need thereof by administering a therapeutically effective amount of a compound described herein. For example, the compounds described herein are effective for treating hematopoietic tumors of lymphoid lineage, leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, Burkett's lymphoma, hematopoietic tumors of myeloid lineage, acute myelogenous leukemias, chronic myelogenous leukemias, myelodysplastic syndrome and promyelocytic leukemia.

The compounds of the present invention are also effective for treating carcinoma of the bladder, carcinoma of the breast, carcinoma of the colon, carcinoma of the kidney, carcinoma of the liver, carcinoma of the lung, small cell lung cancer, esophageal cancer, gall bladder cancer, ovarian cancer, pancreatic cancer, stomach cancer, cervical cancer, thyroid cancer, prostate cancer, skin cancer, squamous cell carcinoma, tumors of mesenchymal origin, fibrosarcoma, rhabdomyosarcoma, tumors of the central and peripheral nervous system, astrocytoma, neuroblastoma, glioma, schwannoma, melanoma, seminoma, teratocarcinoma, osteosarcoma, xenoderoma pigmentosum, keratoctanthoma, thyroid follicular cancer and Kaposi's sarcoma. For example, the compounds of the present invention are effective for treating carcinoma of the breast, ovarian cancer, carcinoma of the liver, carcinoma of the lung, small cell lung cancer, esophageal cancer, gall bladder cancer, ovarian cancer, pancreatic cancer and stomach cancer.

Yet another embodiment is a method of treating leukemia in a patient in need thereof by administering a therapeutically effective amount of a compound of the present invention. For example, the compounds of the present invention are effective for treating acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia, adult T-cell leukemia, and clonal eosinophilias.

### BRIEF DESCRIPTION OF THE FIGURES

FIG. 1 shows the DSC diffractogram of Compound 1A as prepared in Example 1A.

FIG. 2 shows the DSC diffractogram of Compound 1B as prepared in Example 1B.

FIG. 3 shows the DSC diffractogram of Compound 1C as prepared in Example 1C, prepared by Method 2.

FIGS. 4A-4D show the DSC diffractograms of Compound 1D prepared by Methods 1-4 in Example 1D, respectively.

FIG. 5 shows the XRPD diffractogram of Compound 1A as prepared in Example 1A.

FIG. 6 shows the XRPD diffractogram of Compound 1B prepared in Example 1B.

FIGS. 7A and 7B show the XRPD diffractograms of Compound 1C prepared by Methods 1 and 2 in Example 1C, respectively.

FIGS. 8A-8D shows the XRPD diffractogram of Compound 1D prepared by Methods 1-4 in Examples 1D, respectively.

FIG. 9 is a bar graph showing inhibition in cancer cell lines (Examples 4 and 5) using Compound 1 and Compound 1D.

FIGS. 10A and 10B are graphs showing the anti-tumor activity of compound 1D (10, 30, and 100 mg/kg), olaparib (30 mg/kg), cisplatin (5 mg/kg once on day 0), compound 1D (30 mg/kg) with cisplatin, and olaparib (30 mg/kg) with cisplatin in a NCI-H69 Xenograft assay, as described in Example 6(a).

FIGS. 11A and 11B are graphs showing the anti-tumor activity of a vehicle, olaparib (75 mg/kg), compound 1D (75 mg/kg), gemcitabine (21 mg/kg), olaparib (75 mg/jg) with gemcitabine (21 mg/kg), and compound 1D (75 mg/kg) with gemcitabine (21 mg/kg) in an OVCAR-3 Xenograft assay, as described in Example 6(b).

### DETAILED DESCRIPTION OF THE INVENTION

As used herein the following definitions shall apply unless otherwise indicated.

Certain of the compounds described herein contain one or more asymmetric centres and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. For instance, intermediate mixtures may include a mixture of isomers in a ratio of about 10:90, 13:87, 17:83, 20:80, or 22:78. Optically active (R)- and (S)-isomers can be prepared using chiral synthons or chiral reagents, or resolved using known techniques.

Additionally, the present invention also includes compounds which differ only in the presence of one or more isotopically enriched atoms, for example, replacement of hydrogen with deuterium or tritium, or the replacement of a carbon by ¹³C- or ¹⁴C-enriched carbon.

The compounds of the present invention may also contain unnatural proportions of atomic isotopes at one or more of atoms that constitute such compounds. For example, the compounds may be radiolabelled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

Pharmaceutically acceptable salts forming part of this invention include, for example, salts derived from acid addition salts where appropriate which are sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates.

In one embodiment, the salt is methane sulfonate. In one embodiment, the salt is 4-methylbenzenesulfonate. In yet another embodiment, the salt is hydrochloride. In yet another embodiment, the salt is benzenesulfonate.

When ranges are used herein for physical properties, such as molecular weight, or chemical properties, such as chemical formulae, all combinations and sub combinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “comprising” (and related terms such as “comprise” or “comprises” or “having” or “including”) includes, but is not limited to, those embodiments, for example, an embodiment of any composition of matter, composition, method, or process, or the like, that “consist of” or “consist essentially of” the described features.

Abbreviations used herein have their conventional meaning within the chemical and biological arts, unless otherwise indicated.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including, but not limited to, disease treatment. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, the terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as that term is used herein encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” or “patient” refers to an animal, such as a mammal, for example a human. The methods described herein can be useful in both human therapeutics and veterinary applications. In some embodiments, the patient is a mammal, and in some embodiments, the patient is human. For veterinary purposes, the term “subject” and “patient” include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The therapeutic methods of the present invention include methods for the treatment of conditions associated with inflammatory cell activation. “Inflammatory cell activation” refers to the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatibility antigens or cell adhesion molecules) in inflammatory cells (including, but not limited to, monocytes, macrophages, T lymphocytes, B lymphocytes, granulocytes (polymorphonuclear leukocytes including neutrophils, basophils, and eosinophils) mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory condition.

“Autoimmune disease” as used herein refers to any group of disorders in which tissue injury is associated with humoral or cell-mediated responses to the body's own constituents.

“Allergic disease” as used herein refers to any symptoms, tissue damage, or loss of tissue function resulting from allergy.

“Dermatitis” as used herein refers to any of a large family of diseases of the skin that are characterized by inflammation of the skin attributable to a variety of etiologies.

References herein to “a method for treating” a disease of condition using a compound are intended to also encompass a compound for use in the treatment of the disease or condition and/or the use of the compound for the manufacture of a medicament for the treatment of the disease or condition.

The free base forms of the compounds described can be prepared by, for example, the methods described in International Publication No. WO 2021/220120.

### Pharmaceutical Compositions

The present invention provides a pharmaceutical composition comprising one or more compounds according to any embodiment described herein and one or more pharmaceutically acceptable carriers or excipients. In one embodiment, the pharmaceutical composition comprises a therapeutically effective amount of one or more compounds according to any embodiment described herein. The pharmaceutical composition may include one or more additional active ingredients as described herein.

Suitable pharmaceutical carriers and/or excipients may be selected from, but not limited to, diluents, fillers, salts, disintegrants, binders, lubricants, glidants, wetting agents, controlled release matrices, colorants, flavorings, buffers, stabilizers, solubilizers, and combinations thereof.

The pharmaceutical compositions of the present invention can be administered alone or in combination with one or more additional active agents. Where desired, the compound(s) described herein and one or more additional agent(s) may be mixed into a preparation or both components may be formulated into separate preparations to use them in combination separately or at the same time.

The compounds and pharmaceutical compositions of the present invention can be administered by any route that enables delivery of the compound(s) to the site of action, such as orally, intranasally, topically (e.g., transdermally), intraduodenally, parenterally (including intravenously, intraarterially, intramuscularally, intravascularally, intraperitoneally or by injection or infusion), intradermally, by intramammary, intrathecally, intraocularly, retrobulbarly, intrapulmonary (e.g., aerosolized drugs) or subcutaneously (including depot administration for long term release e.g., embedded-under the-splenic capsule, brain, or in the cornea), sublingually, anally, rectally, vaginally, or by surgical implantation (e.g., embedded under the splenic capsule, brain, or in the cornea).

The compositions can be administered in solid, semi-solid, liquid or gaseous form, or may be in dried powder, such as lyophilized form. The pharmaceutical compositions can be packaged in forms convenient for delivery, including, for example, solid dosage forms such as capsules, sachets, cachets, gelatins, papers, tablets, suppositories, pellets, pills, troches, and lozenges. The type of packaging will generally depend on the desired route of administration. Implantable sustained release formulations are also contemplated, as are transdermal formulations.

The amount of the compound of the present invention to be administered is dependent on the subject (e.g., mammal such as human) being treated, the severity of the disorder or condition, the rate of administration, the disposition of the compound and the discretion of the prescribing physician. An effective dosage may be in the range of about 0.001 to about 100 mg per kg body weight per day, such as about 1 to about 35 mg/kg/day, in single or divided doses. For a 70 kg human, this would amount to about 0.05 to 7 g/day, preferably about 0.05 to about 2.5 g/day. An effective amount of a compound of the present invention may be administered in either single or multiple doses (e.g., two or three times a day). The compounds described herein may be formulated using propylene glycol and methyl cellulose and administered to animals.

The compounds of the present invention may be used in combination with one or more anti-cancer agents (e.g., chemotherapeutic agents), therapeutic antibodies, and radiation treatment.

The compounds of the present invention may be formulated or administered in conjunction with additional active agents that act to relieve the symptoms of inflammatory conditions such as encephalomyelitis, asthma, and the other diseases described herein. These additional active agents include non-steroidal anti-inflammatory drugs (NSAIDs).

Preparations of various pharmaceutical compositions are known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999), all of which are incorporated by reference herein in their entirety.

An effective amount of a compound of the present invention may be administered in either single or multiple doses by any of the accepted modes of administration of agents having similar utilities, including, for example, rectal, buccal, intranasal and transdermal routes, by intra-arterial injection, intravenously, intraperitoneally, parenterally, intramuscularly, subcutaneously, orally, topically, or as an inhalant.

### Method of Treatment

The present invention also provides methods of using the compounds or pharmaceutical compositions according to any of the embodiments described herein to treat disease conditions, including, but not limited to, diseases associated with malfunctioning of one or more types of PARP. Conditions and disorders mediated by PARP activity are described in, for example, International Publication Nos. WO 00/42040, WO 01/016136, WO 02/036576, WO 02/090334, WO 03/093261, WO 03/106430, WO 04/080976, WO 04/087713, WO 05/012305, WO 05/012524, WO 05/012305, WO 05/012524, WO 05/053662, WO 06/033003, WO 06/033007, WO 06/033006, WO 06/021801, WO 06/067472, WO 07/144637, WO 07/144639, WO 07/144652, WO 08/047082, WO 08/114114, WO 09/050469, WO 11/098971, WO 15/108986, WO 16/028689, WO 16/165650, WO 17/153958, WO 17/191562, WO 17/123156, WO 17/140283, WO 18/197463, WO 18/038680 and WO 18/108152, all of which are incorporated herein by reference in their entireties.

The treatment methods provided herein comprise administering to the subject a therapeutically effective amount of a compound of the invention. In one embodiment, the present invention provides a method of treating an inflammation disorder, including autoimmune diseases in a mammal. The method comprises administering to the mammal a therapeutically effective amount of a compound of the present invention.

In certain embodiments, the cancer or cancers treatable with the methods provided herein include, but are not limited to:
leukemias, including, but not limited to, acute leukemia, acute lymphocytic leukemia, acute myelocytic leukemias such as myeloblasts, promyelocyte, myelomonocytic, monocytic, erythroleukemia leukemias and myelodysplastic syndrome or a symptom thereof (such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia, and chronic myelomonocytic leukemia (CMML);
chronic leukemias, including, but not limited to, chronic myelocytic (granulocytic) leukemia, chronic lymphocytic leukemia, and hairy cell leukemia;
polycythemia vera;
lymphomas, including, but not limited to, Hodgkin's disease and non-Hodgkin's disease;
multiple myelomas, including, but not limited to, smoldering multiple myeloma, nonsecretory myeloma, osteosclerotic myeloma, plasma cell leukemia, solitary plasmacytoma, and extramedullary plasmacytoma;
Waldenstrom's macroglobulinemia;
monoclonal gammopathy of undetermined significance;
benign monoclonal gammopathy;
heavy chain disease;
bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, and synovial sarcoma;
brain tumors, including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma;
breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer;
adrenal cancer, including, but not limited to, pheochromocytom and adrenocortical carcinoma;
thyroid cancer, including, but not limited to, papillary or follicular thyroid cancer, medullary thyroid cancer, and anaplastic thyroid cancer;
pancreatic cancer, including, but not limited to, insulinoma, gastrinoma, glucagonoma, vipoma, somatostatin-secreting tumor, and carcinoid or islet cell tumor;
pituitary cancer, including, but limited to, Cushing's disease, prolactin-secreting tumor, acromegaly, and diabetes insipidus;
eye cancer, including, but not limited, to ocular melanoma such as iris melanoma, choroidal melanoma, and cilliary body melanoma, and retinoblastoma;
vaginal cancer, including, but not limited to, squamous cell carcinoma, adenocarcinoma, and melanoma;
vulvar cancer, including, but not limited to, squamous cell carcinoma, melanoma, adenocarcinoma, basal cell carcinoma, sarcoma, and Paget's disease;
cervical cancers, including, but not limited to, squamous cell carcinoma, and adenocarcinoma;
uterine cancer, including, but not limited to, endometrial carcinoma and uterine sarcoma;
ovarian cancer, including, but not limited to, ovarian epithelial carcinoma, borderline tumor, germ cell tumor, and stromal tumor;
esophageal cancer, including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma;
stomach cancer, including, but not limited to, adenocarcinoma, fungating (polypoid), ulcerating, superficial spreading, diffusely spreading, malignant lymphoma, liposarcoma, fibrosarcoma, and carcinosarcoma;
colon cancer;
rectal cancer;
liver cancer, including, but not limited to, hepatocellular carcinoma and hepatoblastoma;
gallbladder cancer, including, but not limited to, adenocarcinoma;
cholangiocarcinomas, including, but not limited to, pappillary, nodular, and diffuse;
lung cancer, including, but not limited to, non-small cell lung cancer, squamous cell carcinoma (epidermoid carcinoma), adenocarcinoma, large-cell carcinoma, and small-cell lung cancer;
testicular cancer, including, but not limited to, germinal tumor, seminoma, anaplastic, classic (typical), spermatocytic, nonseminoma, embryonal carcinoma, teratoma carcinoma, and choriocarcinoma (yolk-sac tumor);
prostate cancer, including, but not limited to, adenocarcinoma, leiomyosarcoma, and rhabdomyosarcoma;
penal cancer;
oral cancer, including, but not limited to, squamous cell carcinoma;
basal cancer;
salivary gland cancer, including, but not limited to, adenocarcinoma, mucoepidermoid carcinoma, and adenoidcystic carcinoma;
pharynx cancer, including, but not limited to, squamous cell cancer and verrucous;
skin cancer, including, but not limited to, basal cell carcinoma, squamous cell carcinoma and melanoma, superficial spreading melanoma, nodular melanoma, lentigo malignant melanoma, and acral lentiginous melanoma;
kidney cancer, including, but not limited to, renal cell cancer, adenocarcinoma,
hypernephroma, fibrosarcoma, and transitional cell cancer (renal pelvis and/or uterer);
Wilms' tumor;
bladder cancer, including, but not limited to, transitional cell carcinoma, squamous cell cancer, adenocarcinoma, and carcinosarcoma; and other cancer, including, not limited to, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas
   See, e.g., Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America.

It will be appreciated that the treatment methods described herein are useful in the fields of human medicine and veterinary medicine. Thus, the subject to be treated may be a mammal, preferably a human, or another animal. For veterinary purposes, subjects include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The present invention also relates to a method of treating a hyperproliferative disorder in a subject that comprises administering to the subject a therapeutically effective amount of a compound of the present invention according to any embodiment described herein. In some embodiments, the method relates to the treatment of cancer such as acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, esophageal, testicular, gynecological, thyroid, CNS, PNS, AIDS-related (e.g., Lymphoma and Kaposi's Sarcoma) or viral-induced cancer. In some embodiments, said method relates to the treatment of a non-cancerous hyperproliferative disorder such as benign hyperplasia of the skin (e. g., psoriasis), restenosis, or prostate (e.g., benign prostatic hypertrophy (BPH)).

### Analytical Methods

Differential Scanning Calorimetry (DSC) was performed on DSC Q20 V24.2 Build 107 or Perkin Elmer DSC 4000. Thermal behavior was observed on subjecting the drug substance to heating rate of 10° C./min and with N₂ flow of 50 mL/min or 10° C./min and with N₂ flow of 20 mL/min. Unless stated otherwise, the DSC patterns disclosed herein were obtained using this method.

Powder X-ray Diffraction was performed on an X-ray powder diffractometer P analytical Xpert 3, CuK alpha radiation (lambda=1.54060 Å), LynxEye detector with active length 5.009 degrees 2-theta, laboratory temperature 25±0.3° C., zero background sample holders. Prior to analysis, the samples were gently ground using a mortar and pestle to obtain a fine powder. The ground sample was adjusted into a cavity of the sample holder and the surface of the sample was smoothed using a cover glass. The following measurement parameters were used for analysis:
Scan range—2.5-50° 2θ
Scan mode—continuous
Step size—0.0130° 2θ
Scan step time—18.87 seconds
   Unless stated otherwise, the DSC patterns disclosed herein were obtained using this method.

### EXAMPLES

The examples and preparations provided below further illustrate and exemplify the methods of preparing compounds of the present invention. It is to be understood that the scope of the present invention is not limited in any way by the scope of the following examples and preparations. In the following examples molecules with a single chiral centre, unless otherwise noted, exist as a racemic mixture. Single enantiomers may be obtained by methods known to those skilled in the art.

### General Procedure for Chiral Separation of Racemic Intermediates and Examples:

Chiral compounds, examples and intermediates which were synthetically obtained in racemic forms can be separated into their pure or enriched enantiomeric form by using suitable preparative chromatographic separation methods. By using such methods, Examples 1, 2 and the intermediates (R)-(+)-3-hydroxy-3-methylindolin-2-one and (S)-(−)-3-hydroxy-3-methylindolin-2-one can be obtained in their respective homochiral forms.

### Method 1

Column: CHIRALCEL OJ-H, (250×30) mm, 5 micron
Mobile Phase: Hexanes/EtOH/MeOH/DEA (80/10/10/0.1 v/v/v/v)
Flow rate: 40 mL/min
Detection: UV 210 nm
Temperature: 25° C.
Feed Conc.: 10 mg/mL
Inj vol: 5 mL (on column: 50 mg)
Runtime: 30 min
Cycle time: 12 min

### Method 2

Column: CHIRALCEL OX-H, (250×30) mm, 5 micron
Mobile Phase: CO₂/Co-Solvent 65/35
Co-Solvent: MeOH/ACN/DEA (50/50/0.3 v/v/v)
Flow rate: 120 mL/min
Detection: UV 260 nm
Temperature: 25° C.
Feed Conc.: 20 mg/mL
Inj vol: 5 mL (on column: 100 mg)
Run time: 20 min
Cycle time: 15 min

### Method 3

Column: CHIRALPAK IH, (250×80) mm, 20 micron (DAC column)
Mobile Phase: Acetonitrile/MeOH (80/20 v/v)
Flow rate: 200 mL/min
Detection: UV 300 nm
Temperature: 25° C.
Feed Conc.: 100 mg/mL
Inj vol: 30 mL (on column: 3.0 g)
Runtime: 10 min
Cycle time: 5 min

### Example 1

### (R)-(+)-4-((5-(3-Hydroxy-3-methyl-2-oxoindolin-1-yl)pyridin-3-yl)methyl)phthalazin-1(2H)-one (Compound 1)

### Step 1: Preparation of 4-((5-(3-Hydroxy-3-methyl-2-oxoindolin-1-yl)pyridin-3-yl)methyl)phthalazin-1(2H)-one

3-hydroxy-3-methyl-1-(5-((3-oxoisobenzofuran-1(3H)-ylidene)methyl)pyridin-3-yl)indolin-2-one (1 g, 2.6 mmol) and hydrazine hydrate (156 mg, 3.12 mmol) were dissolved in THE (15 Vols). This mixture was stirred at rt for 1 h. After 1 h acetic acid (78 mg, 1.3 mmol) was added and reaction mixture refluxed at 80° C. Progress of the reaction was monitored by TLC. After completion of the reaction, reaction mixture diluted with water and extracted with MeOH and DCM (1:9) mixture. Organic layer dried on anhydrous Na₂SO₄ and distilled to obtain a crude. Crude was purified by combi-flash or column chromatography using suitable mixture of MeOH and DCM. Purification: Combi-Flash. Eluent: MeOH and DCM: 5.3:94.7. Appearance: Off-White solid. Yield: 236 mg. % Yield: 23%. M.P.: 110-113° C. 1H-NMR (δ ppm, DMSO-d6, 400 MHz): 12.59 (s, 1H), 8.65 (s, 1H), 8.52 (s, 1H), 8.26 (d, J 7.8, 1H), 8.06 (d, J 8.1, 1H), 7.93 (t, J 8, 1H), 7.85 (t, J 8, 1H), 7.79 (s, 1H), 7.43 (d, J 7.2, 1H), 7.21 (t, J 7.6, 1H), 7.11 (t, J 7.2, 1H), 6.69 (d, J 7.7, 1H), 6.12 (s, 1H), 4.46 (s, 2H), 1.48 (s, 3H). MS (m/z): 399.29 ([M+H]+).

### Step 2: Preparation of (R)-(+)-4-((5-(3-Hydroxy-3-methyl-2-oxoindolin-1-yl)pyridin-3-yl)methyl)phthalazin-1(2H)-one

### Method 1:

Using the preparative methods reported in the General Procedure, Method 1, the title compound was resolved as a pure enantiomer from the racemic mixture of 4-((5-(3-hydroxy-3-methyl-2-oxoindolin-1-yl)pyridin-3-yl)methyl)phthalazin-1(2H)-one (1 g). Yield: 381 mg. M.P.: 97-100° C. HPLC Chemical Purity: 99.53%. Chiral Purity: 99.16 (RT: 11.48 mins). ¹H-NMR (δ ppm, DMSO-d6, 400 MHz): 12.58 (s, 1H), 8.65 (d, J 1.7, 1H), 8.52 (d, J 2.2, 1H), 8.26 (d, J 7.4, 1H), 8.06 (d, J 8.0, 1H), 7.93 (t, J 7.8, 1H), 7.85 (t, J 7.8, 1H), 7.79 (t, J 2.0, 1H), 7.43 (d, J 6.9, 1H), 7.22 (t, J 7.8, 1H), 7.11 (t, J 7.6, 1H), 6.69 (d, J 7.6, 1H), 6.12 (s, 1H), 4.47 (s, 2H), 1.48 (s, 3H). MS (m/z): Calcd for M(C₂₃H₁₈N₄O₃); Observed 399.39 ([M+H]⁺).

### Method 2:

Using the preparative method reported in the General Procedure, Method 2, the title compound was resolved as a pure enantiomer from the racemic mixture of 4-((5-(3-hydroxy-3-methyl-2-oxoindolin-1-yl)pyridin-3-yl)methyl)phthalazin-1(2H)-one (48 g). Yield: 15.1 g. M.P.: 227-229° C. DSC: 246.24° C. (Δ 79.57 J/g). HPLC Chemical Purity: 99.56%. Chiral Purity: 99.18 (RT: 12.46 mins). ¹H-NMR (δ ppm, DMSO-d6, 400 MHz): 12.59 (s, 1H), 8.65 (d, J 2.0, 1H), 8.52 (d, J 2.0, 1H), 8.26 (d, J 7.2, 1H), 8.06 (d, J 7.6, 1H), 7.94 (t, J 7.6, 1H), 7.85 (t, J 7.6, 1H), 7.79 (t, J 2.0, 1H), 7.42 (d, J 7.6, 1H), 7.22 (t, J 7.6, 1H), 7.11 (t, J 7.6, 1H), 6.69 (d, J 7.6, 1H), 6.12 (s, 1H), 4.47 (s, 2H), 1.48 (s, 3H). MS (m/z): Calcd for M(C₂₃H₁₈N₄O₃); Observed 399.37 ([M+H]⁺). [α]_{D}²⁵: +30.36° [MeOH:CHLOROFORM (1:9); c 1.0].

### Method 3:

### Step 1: (R)-1-(5-(1,3-dioxolan-2-yl)pyridin-3-yl)-3-hydroxy-3-methylindolin-2-one

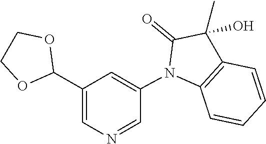 [SMILES: C[C@]1(O)C(=O)N(c2cncc(C3OCCO3)c2)c2ccccc21]

3-bromo-5-(1,3-dioxolan-2-yl)pyridine (88 g, 382 mmol), (R)-3-hydroxy-3-methylindolin-2-one (obtained by chiral resolution from the racemic compound using a chiral prep column general preparatory method-3) (49.9 g, 306 mmol), trans-4-Hydroxy-L-proline (20.0 g, 153 mmol) and potassium carbonate (52.8 g, 382) were dissolved in DMSO (528 ml) and degassed with nitrogen for 30 mins. Copper(I) iodide (14.5 g, 76.5 mmol) was added to the above mixture and again degassed for 30 mins. After degassing, reaction mixture heated to 130° C. and stirred for 4 h at the same temperature. After completion of the reaction, reaction mixture diluted with water (2.9 litre) and extracted with 10% methanol in dichloromethane (3×880 ml). The combined organic layer was washed with 5% aqueous ammonia solution (2×1.3 litre), water (2×1.3 litre), dried over anhydrous sodium sulphate. Evaporation of organic layer on rotavapor afforded the titled compound as a brown gel (78.9 g). Yield: 66%. Compound was taken to the next step without any characterization.

### Step 2: (R)-5-(3-hydroxy-3-methyl-2-oxoindolin-1-yl)nicotinaldehyde

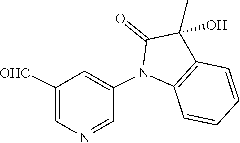 [SMILES: C[C@]1(O)C(=O)N(c2cncc(C=O)c2)c2ccccc21]

(R)-1-(5-(1,3-dioxolan-2-yl)pyridin-3-yl)-3-hydroxy-3-methylindolin-2-one (75 g, 240 mmol) was dissolved in a mixture of water (75 ml) and acetone (75 ml). To this mixture oxalic acid hydrate (150 g, 1.2 mol) was added and stirred at 55° C. for 6 h. Acetone was distilled from the reaction mixture, basified with 10% aq. sodium bicarbonate solution (300 ml) and extracted with ethyl acetate (3×100 ml). The organic layer was washed with water (2×100 ml), dried over anhydrous sodium sulphate and the solvent distilled under vacuum using rotavapor to obtain the crude. To the crude product, isopropanol (10 ml) was added and heated to 60° C. After 30 min. cooled to room temperature, n-hexane (50 ml) was added and stirred for 16 h. The solid precipitated was filtered, washed with isopropanol in n-Hexane (10 ml) and dried under vacuum to afford the titled compound as a pale-brown solid. (30 g). Yield: 47%. ¹H-NMR (S ppm, DMSO-d6, 400 MHz): 10.18 (s, 1H), 9.13 (d, J 1.6, 1H), 8.96 (d, J 2.4, 1H), 8.34 (t, J 2.0, 1H), 7.49 (d, J 7.2, 1H), 7.30 (t, J 7.6, 1H), 7.18 (t, J 7.2, 1H), 6.89 (d, J 8.0, 1H), 6.19 (s, 1H), 1.53 (s, 3H). MS (m/z): 268.9 ([M+H]⁺).

### Step 3: (R)-3-hydroxy-3-methyl-1-(5-((3-oxoisobenzofuran-1(3H)-ylidene)methyl) pyridin-3-yl) indolin-2-one

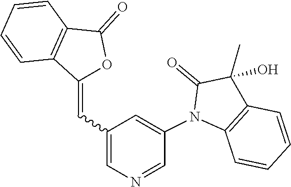 [SMILES: C[C@]1(O)C(=O)N(c2cncc(C=C3OC(=O)c4ccccc43)c2)c2ccccc21]

To (R)-5-(3-hydroxy-3-methyl-2-oxoindolin-1-yl)nicotinaldehyde (35 g, 130 mmol) and (3-oxo-1,3-dihydroisobenzofuran-1-yl)triphenylphosphonium bromide (68.2 g, 143 mmol.) in dichloromethane (350 ml), triethyl amine (36.4 ml, 260 mmol) was added at room temperature. After 1 h, the reaction mixture diluted with dichloromethane (350 ml), washed with water (2×350 ml), dried over anhydrous sodium sulphate and the solvent was evaporated under vacuum to obtain the titled compound as a brown gel (50 g). Yield: >100%. Compound was taken to the next step without any characterization.

### Step 4: (R)-4-((5-(3-hydroxy-3-methyl-2-oxoindolin-1-yl)pyridin-3-yl)methyl)phthalazin-1(2H)-one

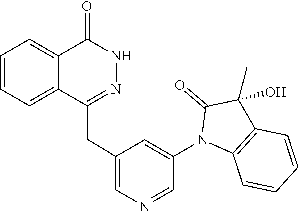 [SMILES: C[C@]1(O)C(=O)N(c2cncc(Cc3n[nH]c(=O)c4ccccc34)c2)c2ccccc21]

(R)-3-hydroxy-3-methyl-1-(5-((3-oxoisobenzofuran-1(3H)-ylidene)methyl)pyridin-3-yl) indolin-2-one (45 g, 117 mmol), hydrazine hydrate (7.56 g, 151 mmol) and 2-Propanol (900 ml) were mixed and refluxed at 100° C. for 3 h. After 3 h, reaction mixture was cooled to room temperature, stirred for 1 h, cooled to 10-15° C. and stirred for 1 h. Water (270 ml) was added to reaction mixture, solid precipitated was filtered and washed with water (2×135 ml). The solid was dissolved in 20% methanol in dichloromethane (5 litre), filtered through cealite bed and distilled under vacuum using rotavap to obtain the residue. The residue was co-distilled with ethyl acetate (2×300 ml) and dried at 90° C. for 12 h to afford the titled compound as an off-white solid (32 g) Yield: 68%. HPLC Chemical Purity: 99.43%. Chiral Purity: 99.90% (RT: 9.10 mins).

### Example 1A

### (R)-(+)-4-((5-(3-Hydroxy-3-methyl-2-oxoindolin-1-yl)pyridin-3-yl)methyl)phthalazin-1(2H)-one, hydrochloride (Compound 1A)

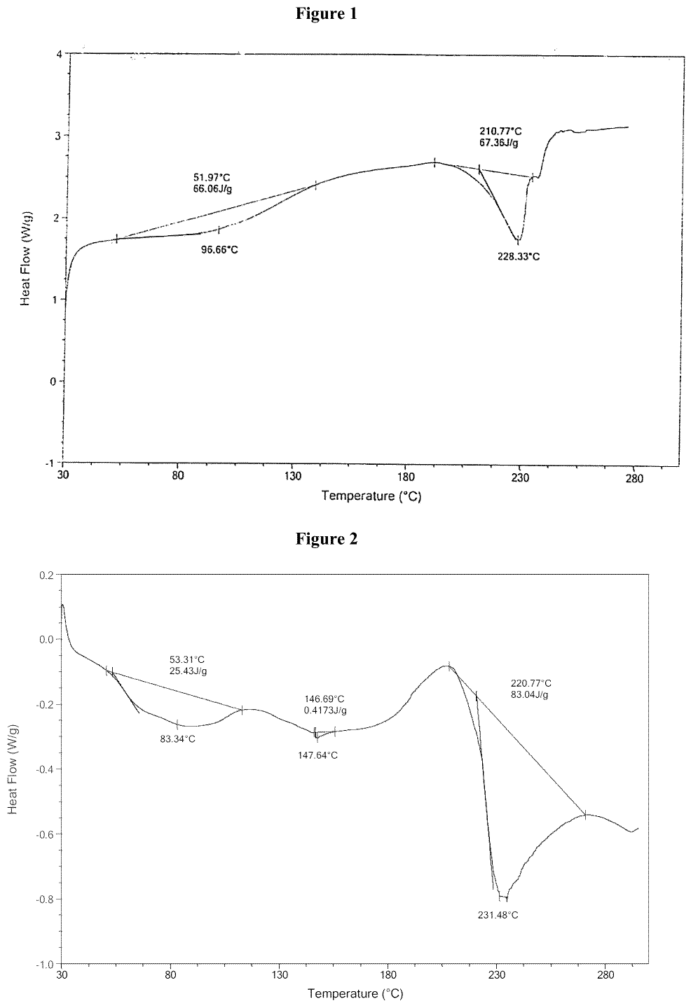

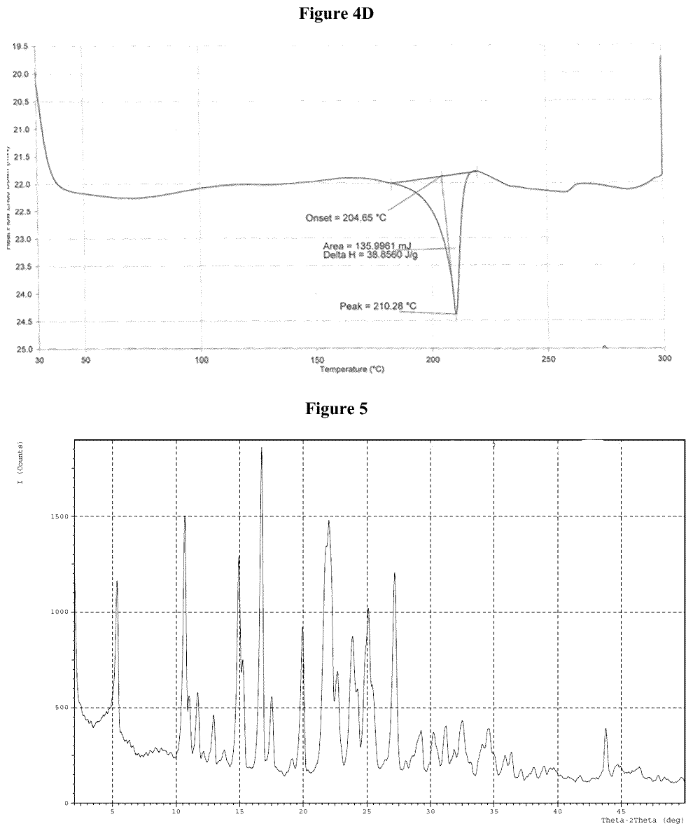

(R)-(+)-4-((5-(3-Hydroxy-3-methyl-2-oxoindolin-1-yl)pyridin-3-yl)methyl)phthalazin-1(2H)-one (6 g, 15.06 mmol) was suspended in MeOH (30 ml). To this mixture Con. HCl (1.73 ml) was added and stirred for 25 mins to obtain a solid. Allowed the reaction mixture to stir 25 more mins at rt and methyl tert-butyl ether (60 ml) was added to the reaction mixture. Stirred the reaction mixture for 2 h and filtered the solid. Solid was dried at 90° C. for 10 h to obtain the titled compound as an off-white solid. Yield: 5.7 g. % Yield: 87%. HCl content by titration: 10.31% (Theoretical: 8.39%). M.P.: 226-228° C. DSC: 228.33° C. (Δ 67.36 J/g). ¹H-NMR (δ ppm, DMSO-d6, 400 MHz): 12.59 (s, 1H), 8.75 (d, J 1.6, 1H), 8.70-8.65 (m, 1H), 8.27 (d, J 7.6, 1H), 8.08 (d, J 8.4, 1H), 8.07-8.02 (m, 1H), 7.95 (td, J 8.4, 1.2, 1H), 7.86 (t, J 8, 1H), 7.45 (d, J 7.2, 1H), 7.24 (td, J 8, 1.2, 1H), 7.13 (t, J 7.2, 1H), 6.79 (dd, J 7.6, 2.4, 1H), 5.93 (bs, —OH and Pyridinium-H), 4.53 (s, 2H), 1.49 (s, 3H). MS (m/z): Calcd for M(C₂₃H₁₈N₄O₃)·HCl; Observed 399.32 ([M+H]⁺). DSC and XRPD diffractograms are provided in FIGS. 1 and 5.

### Example 1B

### (R)-(+)-4-((5-(3-hydroxy-3-methyl-2-oxoindolin-1-yl)pyridin-3-yl)methyl)phthalazin-1(2H)-one, benzene sulfonate (Compound 1B)

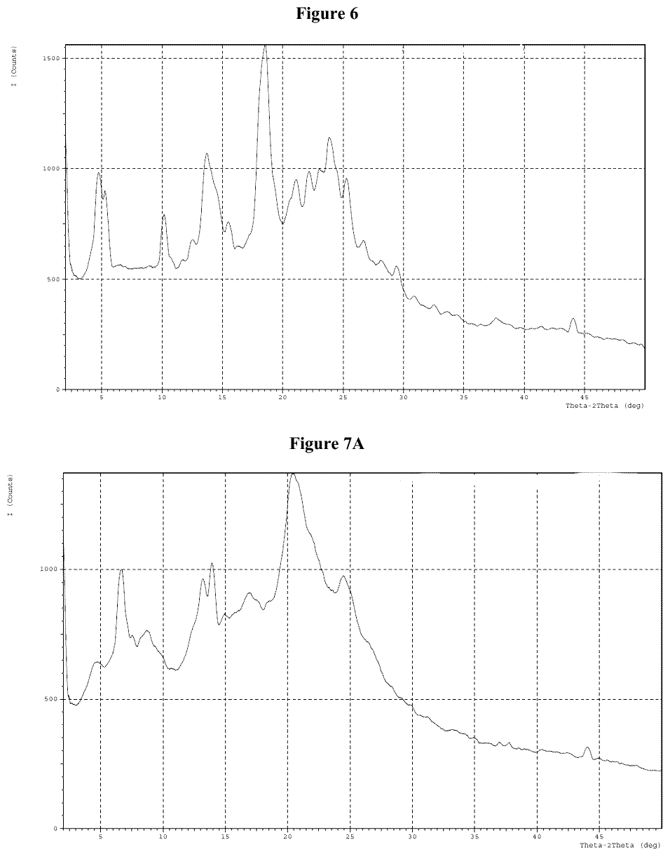

(R)-(+)-4-((5-(3-Hydroxy-3-methyl-2-oxoindolin-1-yl)pyridin-3-yl)methyl)phthalazin-1(2H)-one (1 g, 2.5 mmol) was suspended in acetone (13 ml). Benzenesulfonic acid (436 mg, 2.76 mmol) was dissolved in acetone (2 ml) and added to the above suspension. This mixture was stirred at 50° C. for 1 h. Reaction mixture cooled to rt and diluted with diisopropyl ether (10 ml) to obtain more solid. This mixture was stirred for 2 h at rt. Filtered the solid and washed with diisopropyl ether (10 ml). Dried the solid under vacuum to obtain the titled compound as a pale-brown solid. Yield: 1.27 g. % Yield: 91%. Benzenesulfonic acid content by titration: 28.39% (Theoretical: 28.41%). M.P.: 130-134° C. ¹H-NMR (δ ppm, DMSO-d6, 400 MHz): 12.59 (s, 1H), 8.75 (bs, 1H), 8.68 (bs, 1H), 8.27 (d, J 8, 1H), 8.07 (d, J 8, 1H), 8.04 (bs, 1H), 7.95 (td, J 7.6, 1.2 1H), 7.86 (t, J 8, 1H), 7.59 (dd, J 8, 2.4, 2H), 7.45 (d, J 7.6, 1H), 7.34-7.28 (m, 3H), 7.24 (td, J 8, 1.2, 1H) 7.13 (t, J 7.6, 1H), 6.77 (d, J 8, 1H), 5.6 (bs, —OH and Pyridinium-H), 4.52 (s, 2H), 1.49 (s, 3H). MS (m/z): Calcd for M(C₂₃H₁₈N₄O₃)·C₆H₅SO₃H; Observed 399.10 ([M+H]⁺). DSC and XRPD diffractograms are provided in FIGS. 2 and 6.

### Example 1C

### (R)-(+)-4-((5-(3-hydroxy-3-methyl-2-oxoindolin-1-yl)pyridin-3-yl)methyl)phthalazin-1(2H)-one, 4-methylbenzene sulfonate (Compound 1C)

### Method 1:

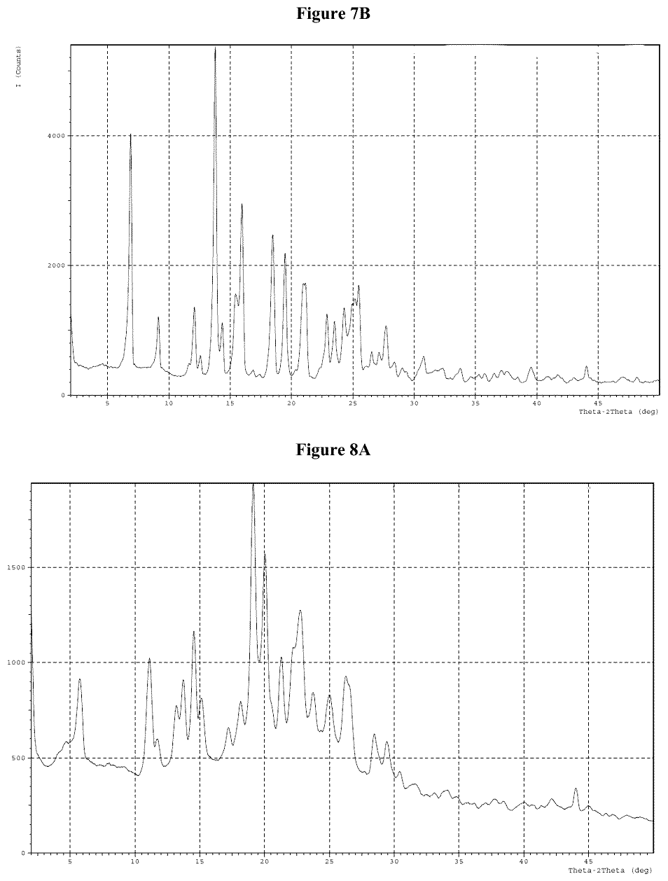

(R)-(+)-4-((5-(3-Hydroxy-3-methyl-2-oxoindolin-1-yl)pyridin-3-yl)methyl)phthalazin-1(2H)-one (2 g, 5 mmol) was suspended in Acetone (23 ml). p-Toluene sulfonic acid monohydrate (900 mg, 5 mmol) dissolved in acetone (4 ml) and added to the above suspension. This heterogeneous mixture was stirred at rt for 1 h. Reaction mixture heated to 70° C. (53° C. inside the reaction vessel) for 3 h. Reaction mixture cooled to rt and stirred at rt for 17 h. Reaction mixture diluted with diisopropyl ether (30 ml) to obtain more solid and stirred at rt for 1 h. Filtered the solid and washed with diisopropyl ether (30 ml). Dried the solid at 90° C. for 1 h. Solid was sieved through 40 mesh and dried at 90° C. for 21 h to obtain the titled compound as a pale-brown solid. Yield: 1.20 g. % Yield: 63%. p-Toluenesulfonic acid content by titration: 30.96% (Theoretical: 30.17%). M.P.: 148-152° C. ¹H-NMR (δ ppm, DMSO-d6, 400 MHz): 12.59 (s, 1H), 8.74 (s, 1H), 8.66 (d, J 2, 1H), 8.27 (d, J 7.6, 1H), 8.07 (d, J 8, 1H), 8.01 (d, J 1.6, 1H), 7.95 (td, J 7.2, 1.2 1H), 7.86 (t, J 7.6, 1H), 7.48-7.43 (m, 3H), 7.24 (td, J 7.6, 1.2, 1H), 7.14 (d, J 7.6, 1H), 7.10 (d, J 8, 2H), 6.76 (d, J 7.6, 1H), 5.01 (bs, —OH and Pyridinium-H), 4.52 (s, 2H), 2.27 (s, 3H), 1.49 (s, 3H). MS (m/z): Calcd for M(C₂₃H₁₈N₄O₃)·C₇H₇SO₃H; Observed 399.35 ([M+H]⁺). XRPD diffractogram is provided in FIG. 7A.

### Method 2:

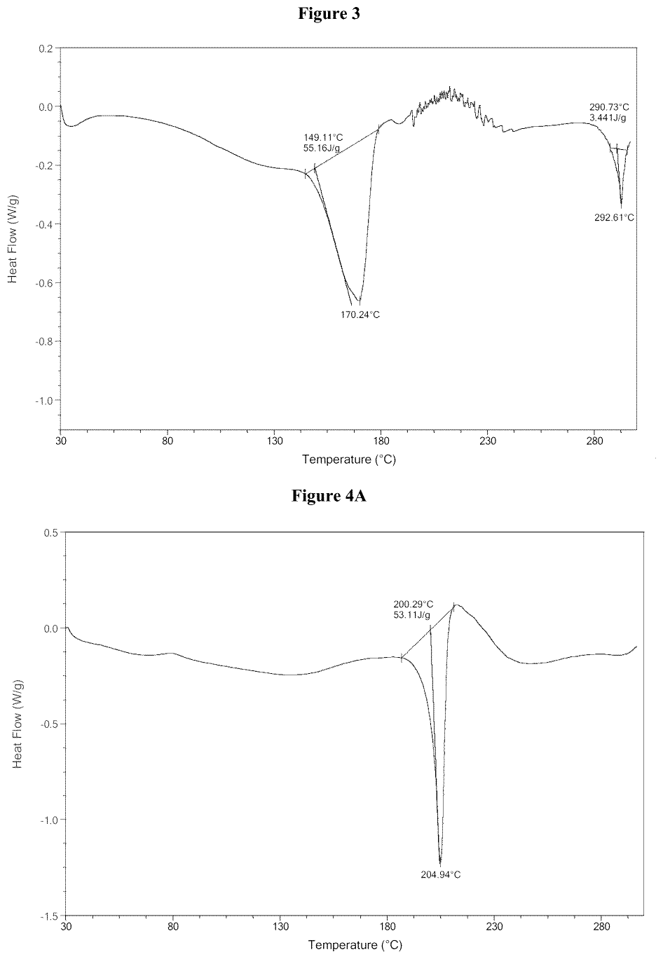

(R)-(+)-4-((5-(3-Hydroxy-3-methyl-2-oxoindolin-1-yl)pyridin-3-yl)methyl)phthalazin-1(2H)-one (2 g, 5 mmol) was suspended in Methanol (22 ml) and heated to 60° C. for 10 mins. At this temperature, p-Toluene sulfonic acid monohydrate (1.05 g, 5 mmol) dissolved in Methanol (2 ml) and added to the above suspension. This heterogeneous mixture was stirred at 60° C. to obtain a clear solution in 7 mins. Reaction mixture stirred at 60° C. for 8 mins. Reaction mixture cooled to rt and solid precipitated at 38° C. inner temperature. Reaction mixture stirred at rt for 1 h. Reaction mixture diluted with methyl tert-butyl ether (24 ml) to obtain more solid and stirred at rt for 2 h. Filtered the solid and washed with methyl tert-butyl ether (30 ml). Dried the solid at 90° C. for 17 h. Solid was sieved through 40 mesh and dried at 90° C. for 8 h to obtain the titled compound as a pale-brown solid. Yield: 2.2 g. % Yield: 77%. p-Toluenesulfonic acid content by titration: 30.79% (Theoretical: 30.17%). M.P.: 174-176° C. DSC: 170.24° C. (Δ 55.16 J/g). ¹H-NMR (δ ppm, DMSO-d6, 400 MHz): 12.60 (s, 1H), 8.74 (s, 1H), 8.67 (d, J 2, 1H), 8.26 (d, J 8, 1H), 8.07 (d, J 8, 1H), 8.02 (t, J 2.4, 1H), 7.95 (t, J 7.2, 1H), 7.86 (t, J 7.6, 1H), 7.46 (d, J 8.4, 3H), 7.24 (t, J 7.6, 1H), 7.14 (d, J 7.6, 1H), 7.10 (d, J 8, 2H), 6.78 (d, J 8, 1H), 5.33 (bs, —OH and Pyridinium-H), 4.52 (s, 2H), 2.27 (s, 3H), 1.49 (s, 3H). MS (m/z): Calcd for M(C₂₃H₁₈N₄O₃)·C₇H₇SO₃H; Observed 399.35 ([M+H]⁺). DSC and XRPD diffractogram are provided in FIG. 3 and FIG. 7B.

### Example 1D

### (R)-(+)-4-((5-(3-Hydroxy-3-methyl-2-oxoindolin-1-yl)pyridin-3-yl)methyl)phthalazin-1(2H)-one, methane sulfonate (Compound 1D)

### Method 1:

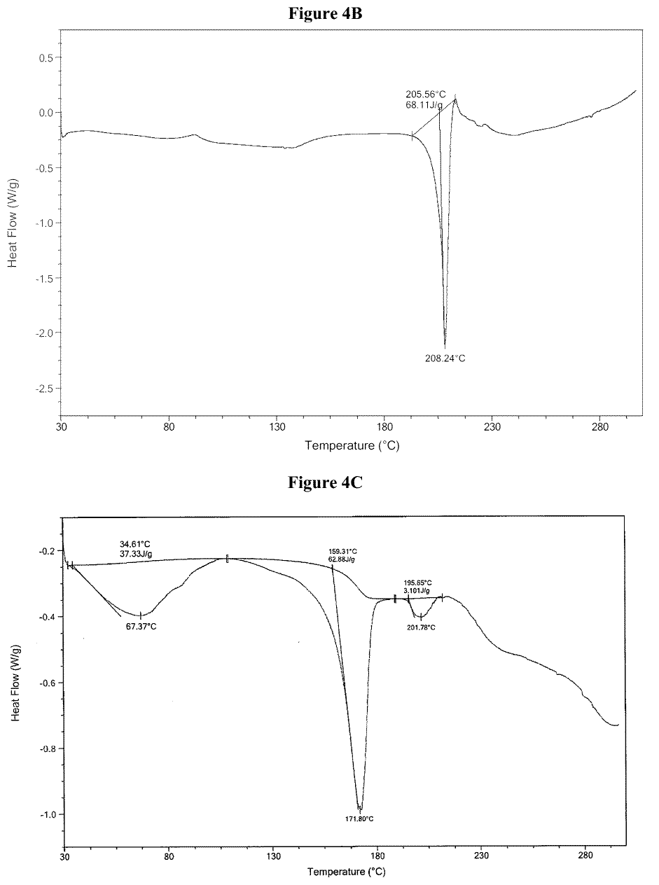

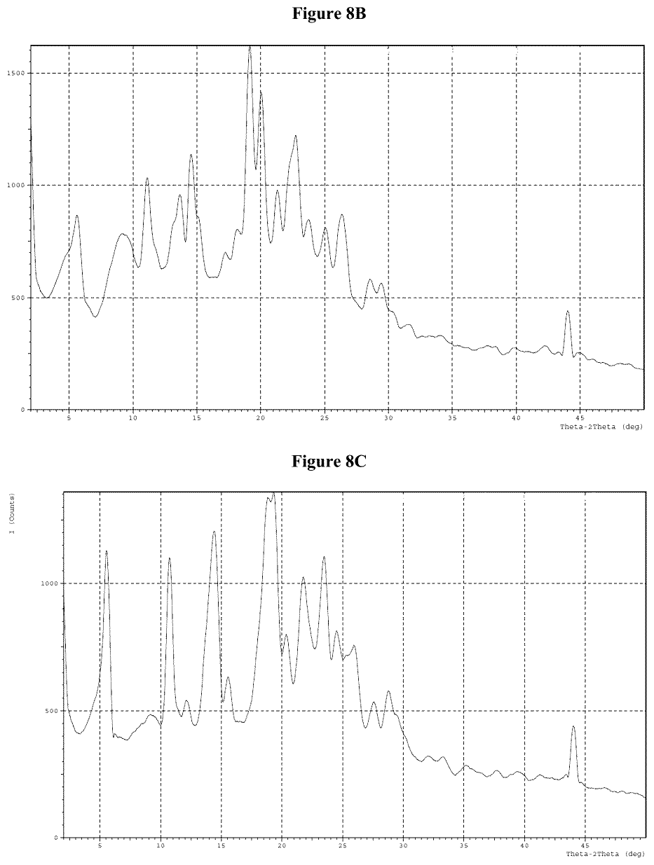

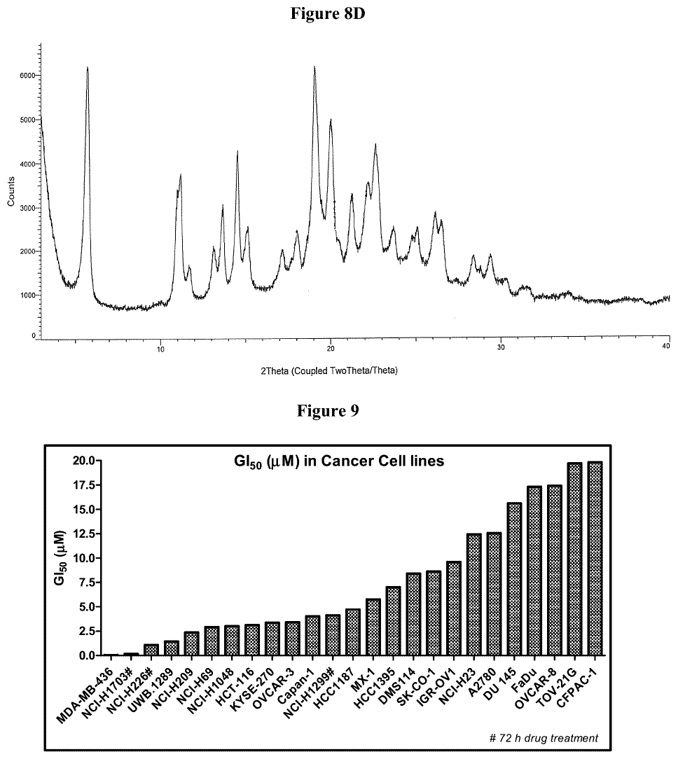

(R)-(+)-4-((5-(3-Hydroxy-3-methyl-2-oxoindolin-1-yl)pyridin-3-yl)methyl)phthalazin-1(2H)-one (1 g, 2.55 mmol) was suspended in Acetone (10 ml). Methanesulfonic acid (300 mg, 3.12 mmol) was added to the above suspension. This mixture was stirred at rt for 10 mins. After 10 mins, reaction mixture warmed to 50° C. (inner temperature 47° C.) and stirred for 1 h. After 1 h, reaction mixture cooled to rt and diluted with diisopropyl ether (10 ml) to obtain more solid. This mixture was stirred for 1 h at rt. Filtered the solid and washed with diisopropyl ether (20 ml). Dried the solid at 90° C. for 17 h. After 17 h, solid was sieved through 40 mesh to obtain the titled compound as a pale-brown solid. Yield: 1.2 g. % Yield: 97%. Methanesulfonic acid content by titration: 19.81% (Theoretical: 19.43%). M.P.: 191-194° C. DSC: 204.94° C. (Δ 53.11 J/g). ¹H-NMR (δ ppm, DMSO-d6, 400 MHz): 12.59 (s, 1H), 8.72 (bs, 1H), 8.68-8.63 (m, 1H), 8.27 (d, J 7.6, 1H), 8.07 (d, J 8, 1H), 8.04-7.98 (m, 1H), 7.95 (td, J 7.2, 1.2, 1H), 7.86 (t, J 8, 1H), 7.45 (d, J 7.2, 1H), 7.24 (t, J 7.6, 1H), 7.13 (t, J 7.6, 1H), 6.75 (d, J 6.8, 1H), 5.54 (bs, —OH and Pyridinium-H), 4.51 (s, 2H), 2.35-2.32 (m, 3H, CH₃SO₃ anion), 1.49 (s, 3H). MS (m/z): Calcd for M(C₂₃H₁₈N₄O₃)·CH₃SO₃H; Observed 399.37 ([M+H]⁺). A DSC and XRPD diffractogram are provided in FIGS. 4A and 8A, respectively

### Method 2:

(R)-(+)-4-((5-(3-Hydroxy-3-methyl-2-oxoindolin-1-yl)pyridin-3-yl)methyl)phthalazin-1(2H)-one (2 g, 5 mmol) was suspended in acetone (20 ml). Methanesulfonic acid (528 mg, 5.5 mmol) was added to the above suspension. This mixture was stirred at rt for 10 mins. After 10 mins, reaction mixture was stirred at 50° C. for 1 h. After 1 h, reaction mixture cooled to rt and diluted with diisopropyl ether (20 ml) to obtain more solid. This mixture was stirred for 1 h at rt. Filtered the solid and washed with diisopropyl ether (20 ml). Dried the solid at 90° C. for 18 h. After 18 h, sold was sieved through 40 mesh. Solid was dried at 90° C. for 2 to obtain the titled compound as Pale-brown solid. Yield: 2.3 g. % Yield: 93%. Methanesulfonic acid content by titration: 19.88% (Theoretical: 19.43%). M.P.: 190-193° C. DSC: 208.24° C. (Δ 68.11 J/g). ¹H-NMR (S ppm, DMSO-d6, 400 MHz): 12.59 (s, 1H), 8.78-8.70 (m, 1H), 8.70-8.62 (m, 1H), 8.26 (d, J 7.6, 1H), 8.07 (d, J 8, 1H), 8.04-7.98 (m, 1H), 7.95 (t, J 7.6, 1H), 7.86 (t, J 7.6, 1H), 7.45 (d, J 7.2, 1H), 7.24 (t, J 7.2, 1H), 7.13 (t, J 7.6, 1H), 6.80-6.74 (m, 1H), 5.59 (bs, —OH and Pyridinium-H), 4.53-4.50 (m, 2H), 2.35-2.31 (m, 3H, CH₃SO₃ anion), 1.49 (s, 3H). MS (m/z): Calcd for M(C₂₃H₁₈N₄O₃)·CH₃SO₃H; Observed 399.31 ([M+H]⁺). A DSC and XRPD diffractogram are provided in FIGS. 4B and 8B, respectively.

### Method 3:

(R)-(+)-4-((5-(3-Hydroxy-3-methyl-2-oxoindolin-1-yl)pyridin-3-yl)methyl)phthalazin-1(2H)-one (15 g, 38 mmol) was suspended in acetone (10 ml). Methanesulfonic acid (4 g, 41 mmol) was added to the above suspension. This mixture was stirred at rt for 10 mins. After 10 mins, reaction mixture warmed to 50° C. (inner temperature 45° C.) and stirred for 1 h. After 1 h, reaction mixture cooled to rt and diluted with diisopropyl ether (150 ml) to obtain more solid. This mixture was stirred for 1 h at rt. Filtered the solid and washed with diisopropyl ether (150 ml). Dried the solid at 90° C. for 5 h. After 5 h, solid was sieved through 40 mesh. Solid was dried at 90° C. for 20 h to obtain the titled compound as a pale-brown solid. Yield: 18 g. % Yield: 97%. Methanesulfonic acid content by titration: 19.77% (Theoretical: 19.43%). M.P.: 190-192° C. DSC: 171.80C (Δ 62.88 J/g). ¹H-NMR (δ ppm, DMSO-d6, 400 MHz): 12.59 (s, 1H), 8.76 (d, J 7.2, 1H), 8.71 (d, J 11.6, 1H), 8.26 (d, J 7.6, 1H), 8.12-8.10 (m, 1H), 8.08 (d, J 8, 1H), 7.95 (t, J 8, 1H), 7.86 (t, J 8, 1H), 7.45 (d, J 7.6, 1H), 7.25 (t, J 8, 1H), 7.14 (t, J 7.6, 1H), 6.79 (t, J 7.6, 1H), 6.41 (bs, —OH and Pyridinium-H), 4.54 (d, J 4.8, 2H), 2.35-2.32 (m, 3H, CH₃SO₃ anion), 1.49 (s, 3H). MS (m/z): Calcd for M(C₂₃H₁₈N₄O₃)·CH₃SO₃H; Observed 397.2 ([M−H]⁻). [α]_{D}²⁵: +15.480 [MeOH:CHLOROFORM (1:9); c 1.0]. DSC and XRPD diffractograms are provided in FIGS. 4C and 8C, respectively.

### Method 4:

(R)-(+)-4-((5-(3-Hydroxy-3-methyl-2-oxoindolin-1-yl)pyridin-3-yl)methyl)phthalazin-1(2H)-one (enantiomeric purity of 92.4:7.6) (433 g, 1.09 mol) was suspended in Acetone (4.3 litre). Methanesulfonic acid (115 g, 1.20 mol) in acetone (216 ml, dissolved at 0° C.) was added to the above suspension. This mixture was stirred at rt for 10 mins. After 10 mins, reaction mixture warmed to 50° C. (inner temperature 45° C.) and stirred for 1 h. After 1 h, reaction mixture cooled to rt and diluted with diisopropyl ether (4.3 litre) to obtain more solid. This mixture was stirred for 1 h at rt. Filtered the solid and washed with diisopropyl ether (2×2.15 litre). Dried the solid at 90° C. for 17 h. The solid was milled to get a fine powder (532 g). To the fine powder, cyclohexane (5.3 litre) was added and heated to reflux for 2 h. After 2 h, cooled to room temperature, filtered and washed with cyclohexane (2.65 litre). Dried the solid at 90° C. for 17 h. The solid was milled to get a fine powder and dried again at 90° C. for 7 h to obtain the titled compound as a Pale-brown solid. Yield: 515 g. % Yield: 95%. HPLC Chemical Purity: 96.42%. Chiral Purity: 90.58% (RT: 9.10 mins). Methanesulfonic acid content by titration: 19.66% (Theoretical: 19.43%). M.P.: 194-198° C. DSC: 210.28° C. (Δ 38.85 J/g). ¹H-NMR (δ ppm, DMSO-d6, 400 MHz): 12.59 (s, 1H), 8.74 (s, 1H), 8.67 (s, 1H), 8.28 (d, J 8.0, 1H), 8.08-8.03 (m, 2H), 7.97 (t, J 7.6, 1H), 7.88 (t, J 7.6, 1H), 7.46 (d, J 7.2, 1H), 7.26 (t, J 7.6, 1H), 7.15 (t, J 7.2, 1H), 6.78 (d, J 7.6, 1H), 5.29 (bs, —OH and Pyridinium-H), 4.52 (s, 2H), 2.34 (s, 3H), 1.49 (s, 3H). MS (m/z): Calcd for M(C₂₃H₁₈N₄O₃)·CH₃SO₃H; Observed 399.37 ([M+H]⁺). DSC and XRPD diffractograms are provided in FIGS. 4D and 8D, respectively.

### Example 2

### (S)-(−)-4-((5-(3-Hydroxy-3-methyl-2-oxoindolin-1-yl)pyridin-3-yl)methyl)phthalazin-1(2H)-one (Compound 2)

### Method 1:

Using the preparative methods reported in the General Procedure, Preparative Method-1 the titled compound was resolved as a pure enantiomer from the racemic mixture of 4-((5-(3-Hydroxy-3-methyl-2-oxoindolin-1-yl)pyridin-3-yl)methyl)phthalazin-1(2H)-one (1 g). Yield: 396 mg. M.P.: 94-97° C. HPLC Chemical Purity: 99.34%. Chiral Purity: 99.82 (RT: 13.61 mins). ¹H-NMR (δ ppm, DMSO-d6, 400 MHz): 12.59 (s, 1H), 8.65 (d, J 1.7, 1H), 8.52 (d, J 2.2, 1H), 8.26 (d, J 7.8, 1H), 8.06 (d, J 7.9, 1H), 7.93 (t, J 7.6, 1H), 7.85 (t, J 8.0, 1H), 7.79 (t, J 2, 1H), 7.43 (d, J 7.2, 1H), 7.21 (t, J 7.8, 1H), 7.11 (t, J 7.8, 1H), 6.69 (d, J 7.8, 1H), 6.12 (s, 1H), 4.46 (s, 2H), 1.48 (s, 3H). MS (m/z): Calcd for M(C₂₃H₁₈N₄O₃); Observed 399.41 ([M+H]⁺). [α]_{D}²⁵: −27.14° [MeOH:CHLOROFORM (1:9); c 1.0].

### Method 2:

Using the preparative methods reported in the General Procedure, Preparative Method-2, the titled compound was resolved as a pure enantiomer from the racemic mixture of 4-((5-(3-hydroxy-3-methyl-2-oxoindolin-1-yl)pyridin-3-yl)methyl)phthalazin-1(2H)-one (85 g). Yield: 37.80 g. M.P.: 229-232° C. HPLC Chemical Purity: 99.37%. Chiral Purity: 99.07 (RT: 13.85 mins). ¹H-NMR (δ ppm, DMSO-d6, 400 MHz): 12.59 (s, 1H), 8.66 (s, 1H), 8.52 (d, J 2, 1H), 8.26 (d, J 7.6, 1H), 8.06 (d, J 8, 1H), 7.94 (t, J 7.6, 1H), 7.85 (t, J 7.6, 1H), 7.80 (s, 1H), 7.44 (d, J 7.2, 1H), 7.22 (t, J 7.6, 1H), 7.11 (t, J 7.6, 1H), 6.69 (d, J 8, 1H), 6.12 (s, 1H), 4.47 (s, 2H), 1.48 (s, 3H). MS (m/z): Calcd for M(C₂₃H₁₈N₄O₃); Observed 399.26 ([M+H]⁺).

### Example 2D

### (S)-(−)-4-((5-(3-Hydroxy-3-methyl-2-oxoindolin-1-yl)pyridin-3-yl)methyl)phthalazin-1(2H)-one, methane sulfonate (Compound 2D)

(S)-(−)-4-((5-(3-Hydroxy-3-methyl-2-oxoindolin-1-yl)pyridin-3-yl)methyl)phthalazin-1(2H)-one (10 g, 25 mmol) was suspended in Acetone (100 ml). Methanesulfonic acid (2.65 g, 27.6 mmol) was added to the above suspension. This mixture was stirred at rt for 10 mins. After 10 mins, reaction mixture was warmed to 50° C. (inner temperature 45° C.) for 1 h. After 1 h, Reaction mixture cooled to rt and diluted with diisopropyl ether (100 ml) to obtain more solid. This mixture was stirred for 1 h at rt. Filtered the solid and dried at 90° C. for 5 h. After 5 h, solid was sieved through 40 mesh. After sieving solid was dried at 90° C. for 20 h. to obtain the titled compound as pale-brown solid. Yield: 12 g. % Yield: 97%. Methanesulfonic acid content by titration: 20.25% (Theoretical: 19.43%). M.P.: 192-194° C. DSC: 205.47° C. (Δ 73.22 J/g). ¹H-NMR (δ ppm, DMSO-d6, 400 MHz): 12.59 (s, 1H), 8.75 (d, J 4.4, 1H), 8.68 (d, J 8.4, 1H), 8.26 (d, J 7.6, 1H), 8.07 (d, J 8, 1H), 8.04-8.00 (m, 1H), 7.95 (t, J 7.6, 1H), 7.86 (t, J 7.6, 1H), 7.45 (d, J 7.2, 1H), 7.24 (t, J 7.6, 1H), 7.13 (t, J 7.6, 1H), 6.77 (t, J 8, 1H), 5.82 (bs, —OH and Pyridinium-H), 4.53 (d, J 3.2, 2H), 2.35-2.32 (m, 3H, CH₃SO₃ anion), 1.49 (s, 3H). MS (m/z; −ve mode): Calcd for M(C₂₃H₁₈N₄O₃)·CH₃SO₃H; Observed 493.1 [M+CH₃SO₃] base peak, 397.3 ([M−H]⁻).

### Pharmacokinetics

### Example 3

The oral bioavailability of Compound 1 and its salts were evaluated in rats. The protocol for the pharmacokinetic (PK) studies in rats is provided below.

### General Methods

Formulation 1: Polysorbate 80 (10% v/v)+Methyl Cellulose (MC) (0.5% w/v)
1. 200 mg^{#}of the test compound was weighed and transferred into a mortar.
2. 1.0 mL of polysorbate 80 (10% v/v of the final suspension) was added to the mortar and the test item was triturated to afford a smooth paste.
3. 9.0 mL of 0.5% methyl cellulose (4000 cps) was added and triturated to afford a fine suspension.
4. The final strength of the formulation was 20.0 mg/mL.
   #For salts of the test compound in order to achieve the equivalent dose level on a molar basis as the free base, a correction factor was used based on the salt and purity.
   Formulation 2: Propylene Glycol (40% v/v)+60% Methyl Cellulose (MC) (0.5% w/v)
1. 1400 mg #of the test compound was weighed and transferred into a glass beaker.
2. 28.0 mL of propylene glycol (1,2-popanediol) (40% v/v of the final volume) was added to the glass beaker and sonicated for 20 min to dissolve. (strength is 50 mg/mL)
3. At the time of dosing, 20 mL of propylene glycol (50.0 mg/mL) and 30.0 mL of 0.5% methyl cellulose (4000 cps) were mixed with continuous stirring to afford a clear solution
4. The final strength of the formulation was 20.0 mg/mL.
#For salts of the test compound in order to achieve the equivalent dose level on a molar basis as the free base, a correction factor was used based on the salt and purity.

Protocol: All animals (mice (BALB/c mice), rats (Wistar rat strains), and dogs (beagle, non-naïve)) were fasted overnight (12 hours) before dosing and continued till 4.0 hours after administration of test item. The blood samples (all collections each of 150 μL from each animal) were collected according to the sampling schedule from orbital sinus or jugular vein into the micro centrifuge tube containing dipotassium EDTA as an anticoagulant. Blood samples were centrifuged immediately with a speed of 4000 RPM for 10 min at 4° C. and separated plasma samples were frozen at below −80° C. and stored until analysis. The plasma concentrations of test item in all samples were analysed by LC-MS/MS method Pharmacokinetic parameters viz. Cₘₐₓ, AUC₀₋ₜ, AUC_{0-∞}, Tₘₐₓ, and t½, were estimated by using WinNonlin software.

TABLE 2
Free base (Compound 1 and Hydrochloride Salt (Compound
1A) in Formulation 1: Rat PK Summary:

| | | Compound 1 | |
|---|---|---|---|
| Compound | | (Prepared by Method 2) | Compound 1A |
| Formulation | | Polysorbate 80 | Polysorbate 80 |
| | | (10% v/v) + | (10% v/v) + |
| | | MC (0.5% w/v) | MC (0.5% w/v) |
| Route | | Oral | Oral |
| Dose | mg/kg | 100 | 100 |
| N | | 3 | 3 |
| Cₘₐₓ | μM | 2.56 | 4.38 |
| AUC₀₋₂₄ | μM · hr | 7.81 | 23.33 |
| Tₘₐₓ | Hr | 0.50 | 0.25 |

TABLE 3
Free base (Compound 1) and Benzene Sulfonate Salt
(Compound 1B) in Formulation 1: Rat PK Summary:

| | | Compound 1 | |
|---|---|---|---|
| Compound | | (Prepared by Method 2) | Compound 1B |
| Formulation | | Polysorbate 80 | Polysorbate 80 |
| | | (10% v/v) + | (10% v/v) + |
| | | MC (0.5% w/v) | MC (0.5% w/v) |
| Route | | Oral | Oral |
| Dose | mg/kg | 100 | 100 |
| N | | 3 | 2 |
| Cₘₐₓ | μM | 2.56 | 17.17 |
| AUC₀₋₂₄ | μM · hr | 7.81 | 32.43 |
| Tₘₐₓ | hr | 0.50 | 0.50 |

TABLE 4
Compound 1 and PTSA Salt (Compound 1C)
in Formulation 1: Rat PK Summary

| | | Compound 1 | Compound 1C | Compound 1C |
|---|---|---|---|---|
| | | (Prepared by | (Prepared by | (Prepared by |
| Compound | | Method 2) | Method 1) | Method 2) |
| Formulation | | Polysorbate 80 | Polysorbate 80 | Polysorbate 80 |
| | | (10% v/v) + | (10% v/v) + | (10% v/v) + |
| | | MC (0.5% w/v) | MC (0.5% w/v) | MC (0.5% w/v) |
| Route | | Oral | Oral | Oral |
| Dose | mg/kg | 100 | 100 | 100 |
| N | | 3 | 3 | 3 |
| Cₘₐₓ | μM | 2.56 | 11.88 | 10.54 |
| AUC₀₋₂₄ | μM · hr | 7.81 | 39.98 | 23.09 |
| Tₘₐₓ | hr | 0.50 | 0.50 | 0.25 |

TABLE 5
Compound 1 and Methane sulfonate Salt (Compound 1D) in Formulation 1 or 2: Rat PK Summary

| | | Compound 1 | Compound 1D | Compound 1D | Compound 1D |
|---|---|---|---|---|---|
| | | (Prepared by | (Prepared by | (Prepared by | (Prepared by |
| Compound | | Method 2) | Method 2) | Method 2) | Method 3) |
| Formulation | | Polysorbate | Polysorbate 80 | PG (40% v/v) + | PG (40% v/v) + |
| | | 80 (10% v/v) + | (10% v/v) + | 60% MC (0.5% w/v) | 60% MC (0.5% w/v) |
| | | MC (0.5% w/v) | MC (0.5% w/v) | | |
| Route | | Oral | Oral | Oral | Oral |
| Dose | mg/kg | 100 | 100 | 100 | 100 |
| N | | 3 | 3 | 3 | 4 |
| Cₘₐₓ | μM | 2.56 | 13.04 | 55.60 | 53.69 |
| AUC₀₋₂₄ | μM · hr | 7.81 | 34.71 | 135.36 | 139.36 |
| Tₘₐₓ | Hr | 0.50 | 0.25 | 0.50 | 0.50 |

TABLE 6
Methane sulfonate Salt (Compound 1D of Method
C): Rat, Mice and Dog PK summary:

| | | Rat | | Mice | | Dog | |
|---|---|---|---|---|---|---|---|
| Route | | IV | Oral | IV | Oral | IV | Oral |
| Dose | mg/kg | 1 | 10 | 1 | 10 | 1 | 20 |
| N | | 4 | 4 | 3 | 3 | 2 | 2 |
| C₀ | μM | 5.25 | — | 3.12 | — | 4.11 | — |
| Cₘₐₓ | μM | 4.47 | 8.43 | 2.87 | 7.62 | 3.47 | 22.87 |
| AUC₀₋₂₄ | μM · hr | 1.18 | 8.96 | 1.12 | 5.64 | 2.73 | 57.05 |
| Tₘₐₓ | hr | — | 0.38 | — | 0.25 | — | 0.50 |

### Biological Assay

Pharmacological properties of the compounds described herein are summarised herein below:

### Example 4

### Cell Proliferation Assay to Determine GI₅₀ in HCT-116, UWB 1.289 and OVCAR-3 Cell Lines (MTT Assay):

### Assay Protocol:

On Day “0” the test cells were plated in 100 μL/well in complete media in a 96-well plate in triplicates and plates were incubated at 37° C. and 5% CO₂. On Day 1, 10 μL of MTT (5 mg/ml) were added to the column designated for Day “0”. It was mixed well and incubated at 37° C. and 5% CO₂ for 3.5 h. Cells were pelleted down at 4000 rpm for 10 minutes. Media was aspirated out and 150 μL of DMSO were added to the cells and mixed by pipetting to dissolve the crystals. Plate was read at A560 nm and A640. DMSO dilutions of the inhibitors (Compound 1 or Compound 1D) were diluted to 3× of required concentration in growth medium. Cells in each well were treated with 50 μL of complete media containing inhibitor. DMSO concentration in the well was 0.1%. Plates were incubated at 37° C. and 5% CO₂ as required for 144 hours. 15 μL of MTT (5 mg/mL) was added to the wells. Plates were incubated at 37° C. and 5% CO₂ for 3.5 hours. After incubation, cells are pelleted down at 4000 rpm for 10 min. Media was aspirated and 150 μL of DMSO per well were added to dissolve the formazan crystals. Plate was read at A560 nm and A640. GI₅₀ values were determined for the compounds of the invention and are shown in FIG. 9.

### Example 5

Cell Proliferation Assay to Determine GI₅₀ in BRCA Mutant and non-BRCA Mutant Cancer Cell Lines:

Cancer cell lines were plated at desired density in their respective complete media and incubated overnight at 37° C. and 5% CO₂. Cells were treated with the different concentrations of the inhibitor for either 72, 120, or 144 hours. At the end of the treatment period, cell viability was measured and GI50 values were calculated.

Compound 1 and Compound 1D inhibited cell growth in both BRCA mutant and non-BRCA mutant cancer cell lines with a GI₅₀ range of 0.043 to 19.83 μM in a dose dependant manner. The results are shown in FIG. 9.

### Example 6

### Anti-Tumor Activity in NCI-H69 and OVCAR-3 Xenografts

### a) NCI-H69 Xenograft:

Each animal female BALB/c nude mice were inoculated with 4×10⁶ NCI-H69 tumor cells (in 0.1 mL, 1:1 with Matrigel) at the right flank by subcutaneous administration under sterile conditions. When the tumors reached an appropriate size (120 mm³), mice were randomized and treatment started. The tumor sizes and animal body weights were measured twice a week. Clinical signs were recorded daily. Test compounds were prepared in Propylene glycol and 0.5% Methyl cellulose (4000 cps). Mice were dosed individually by the most recent body weight. The tumors were measured using a caliper in two dimensions, length (a), and width (b). Tumor volumes were estimated from measurements of the two diameters of the individual tumors as follows: Tumor Volume (mm3)=(a×b²)/2. Tumor Growth Inhibition (TGI) was calculated after each tumor volume measurement according to the formula: % TGI=(TVcn−TVtn)/TVcn×100, where TVtn and TVcn are the mean tumor Volume of treated and control groups, respectively.

### Study Design:

TABLE 8

| | | | Dose | | Treatment |
|---|---|---|---|---|---|
| Group | Animals | Treatment | (mg/kg) | Route | Schedule |
| 1 | 8 | Vehicle | 10 ml | PO | BID × 28 |
| 2 | 8 | 1D | 10 | PO | BID × 28 |
| 3 | 8 | 1D | 30 | PO | BID × 28 |
| 4 | 8 | 1D | 100 | PO | BID × 28 |
| 5 | 8 | Olaparib | 30 | PO | BID × 28 |
| 6 | 8 | Cisplatin | 5 | IP | Once on day 0 |
| 7 | 8 | 1D | 30 | PO | BID × 28 |
| | | Cisplatin | 5 | IP | Once on day 0 |
| 8 | 8 | Olaparib | 30 | PO | BID × 28 |
| | | Cisplatin | 5 | IP | Once on day 0 |

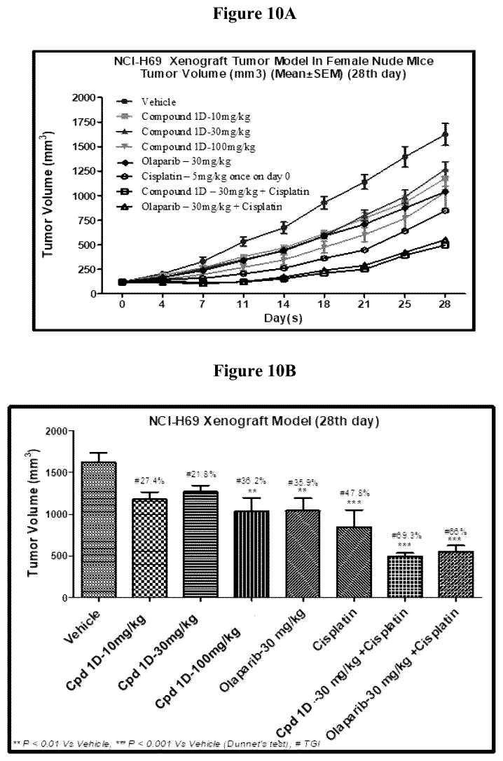

Results: The compounds of the present invention exhibited anti-tumor potential with tumor growth inhibition of 36.200 as a single agent at 100 mg/kg. The results are shown in FIGS. 10A and 10B.

### b) OVACR-3 Xenograft:

Each animal was inoculated with 1×10⁷ OVCAR-3 tumor cells (in 0.1 mL, 1:1 with Matrigel) at the right flank by S.C. administration under sterile conditions. When the tumors reached an appropriate size (100-200 mm3), mice were randomized and treatment started. The tumor sizes and animal body weights were measured twice a week. Clinical signs were recorded daily. Mice were dosed individually by the most recent body weight. The tumors were measured using a caliper in two dimensions, length (a), and width (b). Tumor volumes were estimated from measurements of the two diameters of the individual tumors as follows: Tumor Volume (mm3)=(a×b²)/2. Tumor Growth Inhibition (TGI) was calculated after each tumor volume measurement according to the formula: % TGI=(TVcn−TVtn)/TVcn×100, where TVtn and TVcn are the mean tumor Volume of treated and control groups, respectively.

### Study Design:

TABLE 9

| | | | Dose | | Treatment |
|---|---|---|---|---|---|
| Group | Animals | Treatment | (mg/kg) | Route | Schedule |
| 1 | 10 | Vehicle | 10 ml | PO | BID × 28 |
| 2 | 10 | Olaparib | 75 | PO | BID × 28 |
| 3 | 10 | 1D | 75 | PO | BID × 28 |
| 4 | 10 | Gemcitabine | 21.5 | IP | BIW × 4 wks |
| 5 | 10 | Olaparib | 75 | PO | BID × 28 |
| | | Gemcitabine | 21.5 | IP | BIW × 4 wks |
| 6 | 10 | 1D | 75 | PO | BID × 28 |
| | | Gemcitabine | 21.5 | IP | BIW × 4 wks |

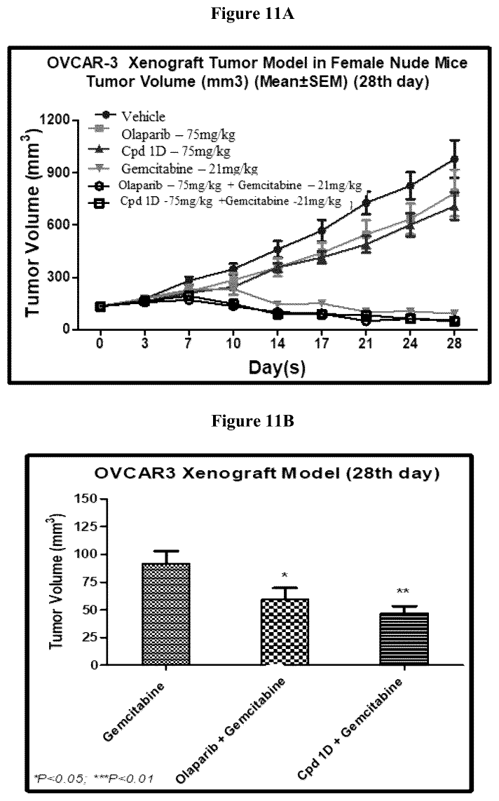

Results: Compounds of the present invention exhibited anti-tumor potential with tumor growth inhibition (TGI) of 28% as a single agent in OVCAR-3 Xenograft model. Combination of the compounds of the present invention with Gemcitabine inhibited the tumor growth significantly (P<0.01) compared to Gemcitabine. The results are shown in FIGS. 11A and 11B.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. It is intended that the appended claims define the scope of the invention and that methods and structures within the scope of these claims and their equivalents be covered thereby.

All publications, patents and patent applications cited in this application are herein incorporated by reference to the same extent as if each individual publication, patent or patent application was specifically and individually indicated to be incorporated herein by reference.

1. A compound selected from
(R)-(+)-4-((5-(3-Hydroxy-3-methyl-2-oxoindolin-1-yl)pyridin-3-yl)methyl)phthalazin-1(2H)-one hydrochloride;
(R)-(+)-4-((5-(3-Hydroxy-3-methyl-2-oxoindolin-1-yl)pyridin-3-yl)methyl)phthalazin-1(2H)-one benzenesulfonate;
(R)-(+)-4-((5-(3-Hydroxy-3-methyl-2-oxoindolin-1-yl)pyridin-3-yl)methyl)phthalazin-1(2H)-one 4-methyl benzenesulfonate; and
(R)-(+)-4-((5-(3-Hydroxy-3-methyl-2-oxoindolin-1-yl)pyridin-3-yl)methyl)phthalazin-1(2H)-one methanesulfonate.

2-5. (canceled)

6. A compound selected from
(S)-(−)-4-((5-(3-Hydroxy-3-methyl-2-oxoindolin-1-yl)pyridin-3-yl)methyl)phthalazin-1(2H)-one hydrochloride;
(S)-(−)-4-((5-(3-Hydroxy-3-methyl-2-oxoindolin-1-yl)pyridin-3-yl)methyl)phthalazin-1(2H)-one benzenesulfonate;
(S)-(−)-4-((5-(3-Hydroxy-3-methyl-2-oxoindolin-1-yl)pyridin-3-yl)methyl)phthalazin-1(2H)-one 4-methyl benzenesulfonate; and
(S)-(−)-4-((5-(3-Hydroxy-3-methyl-2-oxoindolin-1-yl)pyridin-3-yl)methyl)phthalazin-1(2H)-one methanesulfonate.

7-11. (canceled)

12. A crystalline hydrochloride salt of (R)-(+)-4-((5-(3-Hydroxy-3-methyl-2-oxoindolin-1-yl)pyridin-3-yl)methyl)phthalazin-1(2H)-one, wherein the crystalline hydrochloride salt exhibits one or more of:
a) an X-ray powder diffraction pattern having one or more characteristic peaks at 5.32, 10.65, 14.91, 15.22, 16.68, 19.90, 21.75, 21.99, 23.84, 25.08, and 27.14±0.2° 2θ;
b) an X-ray powder diffraction pattern substantially as depicted in FIG. 5;
c) a differential scanning calorimeter pattern with a characteristic endothermic peak of about 228° C.; or
d) a differential scanning calorimeter pattern substantially as depicted in FIG. 1.

13-17. (canceled)

18. A crystalline benzenesulfonate salt of (R)-(+)-4-((5-(3-Hydroxy-3-methyl-2-oxoindolin-1-yl)pyridin-3-yl)methyl)phthalazin-1(2H)-one, wherein the crystalline benzenesulfonate salt exhibits one or more of:
(a) an X-ray powder diffraction pattern having one or more characteristic peaks at 4.91, 5.42, 13.76, 14.61, 18.47, 21.14, 22.19, 23.07, 23.84, and 25.28±0.2° 2θ;
(b) an X-ray powder diffraction pattern substantially as depicted in FIG. 6;
(c) a differential scanning calorimeter pattern with a characteristic endothermic peak of about 231° C.; or
(d) a differential scanning calorimeter pattern substantially as depicted in FIG. 2.

19-23. (canceled)

24. A crystalline mono-methylbenzene sulfonate salt of (R)-(+)-4-((5-(3-Hydroxy-3-methyl-2-oxoindolin-1-yl)pyridin-3-yl)methyl)phthalazin-1(2H)-one, wherein the crystalline mono-methylbenzene sulfonate salt exhibits one or more of:
(a) an X-ray powder diffraction pattern having one or more peaks at 6.81, 13.22, 13.96, 20.52 21.87, 22.67, and 24.48±0.2° 2θ;
(b) an X-ray powder diffraction pattern substantially as depicted in FIG. 7A;
(c) an X-ray powder diffraction (XRPD) pattern having one or more characteristic peaks at 6.98, 13.82, 15.98, 18.50, and 19.50±0.2° 2θ;
(d) an XRPD pattern substantially as depicted in FIG. 7B;
(e) a differential scanning calorimeter (DSC) pattern with a characteristic endothermic peak at about 170° C.;
(f) a differential scanning calorimeter (DSC) pattern substantially as depicted in FIG. 3; or
(g) an XRPD pattern exhibiting one or more peaks selected from 6.98, 13.82, 15.98, 18.50, 19.50±0.05, 0.1, or 0.2° 2θ, and a differential scanning calorimeter (DSC) pattern having a characteristic endothermic peak at about 170° C.

25-31. (canceled)

32. A crystalline methane sulfonate salt of (R)-(+)-4-((5-(3-Hydroxy-3-methyl-2-oxoindolin-1-yl)pyridin-3-yl)methyl)phthalazin-1(2H)-one, wherein the crystalline methane sulfonate salt exhibits one or more of:
(a) an X-ray powder diffraction pattern having one or more characteristic peaks at 5.84, 11.17, 13.78, 14.60, 19.17, 20.03, 21.32, 22.24, 22.77, 26.40±0.2° 2θ;
(b) an X-ray powder diffraction pattern substantially as depicted in FIG. 8A;
(c) an X-ray powder diffraction pattern having one or more characteristic peaks at 5.74, 11.20, 13.69, 14.67, 19.20, 20.05, 21.29, 22.57, 26.38±0.2° 2θ;
(d) an X-ray powder diffraction pattern substantially as depicted in FIG. 8B;
(e) an X-ray powder diffraction pattern having one or more characteristic peaks at 5.67, 10.81, 14.34, 19.03, 20.40, 21.96, 23.44, 24.52, and 25.94±0.2° 2θ;
(f) an X-ray powder diffraction pattern substantially as depicted in FIG. 8C;
(g) an X-ray powder diffraction pattern having one or more peaks at 5.73, 11.02, 11.19, 13.68, 14.55, 15.11, 19.11, 20.04, 21.28, 22.19, and 22.65, 26.15±0.2° 2θ;
(h) an X-ray powder diffraction pattern substantially as depicted in FIG. 8D;
(i) a differential scanning calorimeter pattern with a characteristic endothermic peak in the range between about 165° C. to 175° C.;
(i) a differential scanning calorimeter pattern with a characteristic endothermic peak in the range between about 190° C. and 220° C.;
(k) a differential scanning calorimeter pattern with a characteristic endothermic peak of about 205° C.;
(l) a differential scanning calorimeter pattern substantially as depicted in FIG. 4A;
(m) exhibits a differential scanning calorimeter pattern with a characteristic endothermic peak of about 208° C.;
(n) a differential scanning calorimeter pattern substantially as depicted in FIG. 4B;
(o) a differential scanning calorimeter pattern with a characteristic endothermic peak of about 172° C.;
(p) a differential scanning calorimeter pattern substantially as depicted in FIG. 4C;
(q) a differential scanning calorimeter pattern with a characteristic endothermic peak of about 210° C.; or
(r) a differential scanning calorimeter pattern substantially as depicted in FIG. 4D.

33-54. (canceled)

55. A pharmaceutical composition comprising a compound according to claim 1 and a pharmaceutically acceptable excipient.

56-58. (canceled)

59. A method of inhibiting a catalytic activity of a PARP enzyme present in a cell comprising contacting the cell with an effective amount of a compound according to claim 1.

60. The method of claim 59, wherein the inhibition takes place in a subject suffering from a disease or disorder which is cancer, a bone disorder, an inflammatory disease, an immune disease, a nervous system disease, a metabolic disease, a respiratory disease, thrombosis, or a cardiac disease.

61-62. (canceled)

63. A method for the treatment of a PARP associated disease or disorder comprising administering to a subject in need thereof an effective amount of a compound according to claim 1.

64. (canceled)

65. The method of claim 63, wherein the PARP associated disease, disorder or condition is an immune system-related disease, a disease or disorder involving inflammation, cancer or other proliferative disease, a hepatic disease or disorder, or a renal disease or disorder.

66. The method of claim 63, wherein the PARP associated disease, disorder or condition is selected from inflammation, glomerulonephritis, uveitis, hepatic diseases or disorders, renal diseases or disorders, chronic obstructive pulmonary disease, rheumatoid arthritis, inflammatory bowel disease, vasculitis, dermatitis, osteoarthritis, inflammatory muscle disease, allergic rhinitis, vaginitis, interstitial cystitis, scleroderma, osteoporosis, eczema, allogeneic or xenogeneic transplantation, graft rejection, graft-versus-host disease, lupus erythematosus, pulmonary fibrosis, dermatomyositis, thyroiditis, myasthenia gravis, autoimmune hemolytic anemia, cystic fibrosis, chronic relapsing hepatitis, primary biliary cirrhosis, allergic conjunctivitis, hepatitis, atopic dermatitis, asthma, Sjogren's syndrome, organ transplant rejection, multiple sclerosis, Guillain-Barre, autoimmune uveitis, pernicious anemia, autoimmune thrombocytopenia, temporal arteritis, antiphospholipid syndrome, vasculitides, Wegener's granulomatosis, Behcet's disease, psoriasis, dermatitis herpetiformis, pemphigus vulgaris, vitiligo, Crohn's disease, colitis, ulcerative colitis, primary biliary cirrhosis, autoimmune hepatitis, Type 1 or immune-mediated diabetes mellitus, Grave's disease, Hashimoto's thyroiditis, autoimmune oophoritis and orchitis, autoimmune disorder of the adrenal gland, systemic lupus erythematosus, polymyositis, dermatomyositis, ankylosing spondylitis, transplant rejection, skin graft rejection, arthritis, bone diseases associated with increased bone resorption, ileitis, Barrett's syndrome, adult respiratory distress syndrome, chronic obstructive airway disease; corneal dystrophy, trachoma, onchocerciasis, sympathetic ophthalmitis, endophthalmitis, gingivitis, periodontitis, tuberculosis, leprosy, uremic complications, nephrosis, sclerodermatitis, psoriasis, chronic demyelinating diseases of the nervous system, AIDS-related neurodegeneration, Alzheimer's disease, infectious meningitis, encephalomyelitis, Parkinson's disease, Huntington's disease, amyotrophic lateral sclerosis viral or autoimmune encephalitis, autoimmune disorders, immune-complex vasculitis, systemic lupus and erythematodes, systemic lupus erythematosus (SLE), cardiomyopathy, ischemic heart disease hypercholesterolemia, atherosclerosis, preeclampsia, chronic liver failure, brain and spinal cord trauma, and cancer.

67. The method of claim 63, wherein the PARP associated disease, disorder or condition is selected from hematopoietic tumors of lymphoid lineage, leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma, hematopoietic tumors of myeloid lineage, acute myelogenous leukemias, chronic myelogenous leukemias, myelodysplastic syndrome, promyelocytic leukemia, carcinoma of the bladder, carcinoma of the breast, carcinoma of the colon, carcinoma of the kidney, carcinoma of the liver, carcinoma of the lung, small cell lung cancer, esophageal cancer, gall bladder cancer, ovarian cancer, pancreatic cancer, stomach cancer, cervical cancer, thyroid cancer, prostate cancer, skin cancer, squamous cell carcinoma, tumors of mesenchymal origin, fibrosarcoma, rhabdomyosarcoma, tumors of the central and peripheral nervous system, astrocytoma, neuroblastoma, glioma, schwannoma, melanoma, seminoma, teratocarcinoma, osteosarcoma, xenoderoma pigmentosum, keratoctanthoma, thyroid follicular cancer and Kaposi's sarcoma.

68. The method of claim 63, wherein the PARP associated disease, disorder or condition is selected from carcinoma of the breast, ovarian cancer, carcinoma of the liver, carcinoma of the lung, small cell lung cancer, esophageal cancer, gall bladder cancer, pancreatic cancer or stomach cancer.

69. The method of claim 63, wherein the PARP associated disease, disorder or condition is carcinoma of the breast or ovarian cancer.
